(19) United States
(12) Patent Application Publication
(10) Pub. No.: US 2025/0064543 A1
(43) Pub. Date: Feb. 27, 2025

(54) STERILE DRAPE FOR DRAPING EQUIPMENT, AND RELATED DEVICES, SYSTEMS AND METHODS

(71) Applicant: INTUITIVE SURGICAL OPERATIONS, INC., Sunnyvale, CA (US)

(72) Inventors: Ryan Abbott, San Jose, CA (US); Bram Gilbert Antoon Lambrecht, Redwood City, CA (US); Kevin Lau, Fremont, CA (US); Steven Manuel, Sunnyvale, CA (US); Ashley L. Oliver, Piedmont, CA (US)

(73) Assignee: INTUITIVE SURGICAL OPERATIONS, INC., Sunnyvale, CA (US)

(21) Appl. No.: 18/724,668

(22) PCT Filed: Dec. 28, 2022
(86) PCT No.: PCT/US2022/082460

§ 371 Date: Jun. 27, 2024

Related U.S. Application Data
(60) Provisional application No. 63/294,138, filed on Dec. 28, 2021.

(51) Int. Cl. A61B 46/10

(52) U.S. Cl. CPC A61B 46/10

(57) ABSTRACT

A sterile drape assembly for draping equipment to create a sterile barrier around the equipment may comprise a hollow elongated drape body and a closure mechanism. The drape body extends between a first end and a second end and has a first drape portion comprising the first end and a second drape portion comprising the second end. The closure mechanism releasably closes, or is configured to releasably close, the first end of the drape body. The sterile drape assembly has a partially inverted state in which the first drape portion is folded back over and covers the second drape portion, and the second drape portion defines a partially enclosed interior volume configured to receive at least a first portion of the equipment.

### CROSS-REFERENCE TO RELATED APPLICATION

This application claims priority to U.S. Provisional Application No. 63/294,138 (filed Dec. 28, 2021), titled “STERILE DRAPE FOR DRAPING EQUIPMENT, AND RELATED DEVICES, SYSTEMS AND METHODS” the entire contents of which are incorporated by reference herein.

### FIELD

Aspects of this disclosure relate generally to sterile drapes for use in draping equipment being used in procedures performed in a sterile environment, and related devices, systems, and methods. In particular, aspects of the disclosure relate to such drapes, and related device, systems, and methods, for use in draping equipment during a medical procedure.

### INTRODUCTION

Various procedures require a sterile field to be established around at least part of an environment or worksite. For example, various medical procedures, such as surgical, diagnostic, therapeutic, or imaging procedures, may require a sterile field in an environment including and within a distance around a patient. Some industrial procedures, for example manufacturing of sensitive electronic components, also may require or benefit from establishing a sterile field. A sterile field is a region in which any exposed surfaces of objects in the region are maintained in a sterile condition (i.e., a condition substantially free from contaminants, such as biological pathogens, dusts, oils, etc.). One way to ensure sterility in the sterile field is to perform a sterilization process, such as applying sterilizing chemicals, heat treatment, etc., on exposed surfaces in the sterile field. Another means of maintaining sterility in the sterile field is to cover non-sterile surfaces with one or more sterile drapes, instead of or in addition to sterilizing the surface. A sterile drape creates a sterile barrier between the environment of the sterile field and the covered surface. An exterior surface of the sterile drape is sterile, and this external surface of the sterile drape is exposed to the environment of the sterile field instead of the covered surface of the object. In this way, the lack of sterility of the covered surface of the object does not compromise the sterility of the sterile field. Sterilization and sterile drapes are often used in conjunction, with some surfaces in the sterile field being sterilized and other surfaces in the sterile field being covered with a sterile drape. Sterile drapes may be particularly advantageous to use, for example, with surfaces that are difficult and/or expensive to sterilize, for example due to their size or shape, or with surfaces which comprise (or are) sensitive components that could be damaged by sterilization processes.

Medical procedures or industrial procedures, such as those described above, may utilize equipment that is difficult to sterilize. By way of example, in some computer-assisted, teleoperated systems, a manipulator system is employed which comprises one or more manipulators that support and provide forces to manipulate an instrument coupled thereto. The manipulators may comprise one or more links, with adjacent links being coupled together by joints that allow relative motion between the links, thereby providing degrees of freedom of motion for the manipulator. An instrument may be removably couplable to the manipulator, and may comprise an end effector with one or more functional elements, such as, for example, a jaw mechanism, a stapler, a cutting implement, a camera, an electrode, a sensor, etc., to perform one or more functions of the instrument, such as cutting, sealing, grasping, imaging, etc. Driving inputs, such as driving forces, electricity, or other inputs, may be supplied from an interface of the manipulator to the mounted instrument to drive degrees of freedom of motion and/or functions of the instrument. The manipulators are movable around a worksite, for example under the remote control of an operator while carrying out a desired procedure. As used herein, a worksite comprises a space around and containing the target of a procedure (e.g., a patient, an article of manufacture, etc.), such as a region around an operating table in a surgical context, a region around a portion of an assembly line in a manufacturing context, etc. In some cases, the sterile field may be coextensive with the worksite, while in other cases the sterile field may extend beyond the worksite, and in still other cases the sterile field may comprise only a portion of the worksite.

When a manipulator system is used in a procedure that needs a sterile field, some equipment of the manipulator system is usually located in the sterile field, and this equipment may need to be sterilized or covered with a sterile drape, as described above. In particular, manipulators will generally be located at least partially in the sterile field. In some systems, the manipulators can also be moved out of the sterile field, for example while a procedure is being initially prepared for, and then be moved into the sterile field once the procedure is underway. Manipulators or other equipment of a manipulator system can be difficult to sterilize, as they may be relatively large, may have moving parts and many nooks and crannies that may be difficult to reach with sterilization procedures, and/or may have sensitive parts that may be damaged by sterilization. Accordingly, manipulators or other equipment often need to be covered by a sterile drape during procedures.

Other pieces of equipment may pose similar challenges, such as support equipment (e.g., Mayo stands and the like in a medical procedure) that holds instruments to be used in a procedure or any piece of equipment that may be difficult to sterilize but that may be moved into and out of a sterile field.

However, in some contexts, it can be challenging to appropriately drape a manipulator or other equipment in a way that maintains sterility of the sterile field while not interfering with the preparation of a worksite for a procedure. A need exists for sterile drapes and draping methods that facilitate and ease sterile draping of a manipulator or other equipment without contaminating the sterile field and without interfering with preparation of the worksite.

### SUMMARY

Various embodiments of the present disclosure may solve one or more of the above-mentioned problems and/or may demonstrate one or more of the above-mentioned desirable features. Other features and/or advantages may become apparent from the description that follows.

In accordance with at least one embodiment of the present disclosure a sterile drape assembly for draping equipment to create a sterile barrier around the equipment comprises a hollow elongated drape body and a closure mechanism. The drape body extends between a first end and a second end, and comprises a first drape portion comprising the first end and a second drape portion comprising the second end. The closure mechanism releasably closes, or is configured to releasably close, the first end of the drape body. In a partially inverted state of the sterile drape assembly, the first drape portion is folded back over and covers the second drape portion, and the second drape portion defines a partially enclosed interior volume configured to receive at least a first portion of the equipment. In some embodiments, the sterile drape assembly also has a fully inverted state. In some embodiments, in the fully inverted state the closure mechanism is released and the first end of the drape body is open, the second end of the drape body is closed, the first and second drape portions of the drape body are positioned sequentially adjacent one another along a length of the drape body and not overlapping, and the partially enclosed internal volume is extended so as to receive at least the first portion and a second portion of the equipment to create a sterile barrier around the first and second portions of the equipment.

In accordance with at least one embodiment of the present disclosure, a sterile drape assembly for draping equipment to create a sterile barrier around the equipment comprises a hollow elongated drape body and a closure mechanism. The drape body comprises a first end, a second end, and a lateral wall extending from the first end to the second end and bounding a first interior volume. The closure mechanism is located at the first end. The drape body is in, or configured to be placed in, a partially inverted state in which a portion of the drape body comprising the first end is folded back over and covers a remaining portion of the drape body comprising the second end. In the partially inverted state, the drape body defines a second interior volume configured to receive a portion of the equipment.

In accordance with at least one embodiment of the present disclosure, a method of draping equipment to maintain a sterile field comprises covering a first portion of the equipment with a sterile drape assembly in a partially inverted state. In the partially inverted state, a first drape portion of the sterile drape assembly is folded back over and covers a second drape portion of the sterile drape assembly to define a first internal volume in which the first portion of the equipment is received. The method further comprises opening a first end of the sterile drape assembly, the first end being part of the first drape portion of the sterile drape assembly. The method further comprises moving the opened first end of the first drape portion along the second drape portion to uncover the first drape portion.

In accordance with at least one embodiment of the present disclosure, a method of manufacturing a sterile drape assembly for draping equipment to create a sterile barrier around the equipment comprises providing a hollow drape body having a first end with a releasable closure mechanism, and a second end opposite from the first end. The method further comprises partially inverting the hollow drape body such that a first drape portion of the hollow drape body covers a second drape portion of the hollow drape body and the first drape portion defines an interior volume configured to receive at least a portion of the equipment, the first drape portion comprising the first end and the second drape portion comprising the second end.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1A is a schematic perspective view of an embodiment of a sterile drape assembly in a first state.

FIG. 1B is a schematic perspective view of the sterile drape assembly of FIG. 1A in a second state.

FIG. 1C is a schematic perspective view of the sterile drape assembly of FIG. 1A installed on a manipulator in the second state.

FIG. 1D is a schematic perspective view of the sterile drape assembly of FIG. 1A installed on the manipulator in a third state.

FIG. 1E is a schematic perspective view of the sterile drape assembly of FIG. 1A installed on the manipulator in fourth state.

FIG. 2A is a cross-section the sterile drape assembly of FIG. 1A, with the section taken along the longitudinal centerline of the sterile drape.

FIG. 2B is a cross-section the sterile drape assembly of FIG. 1B in the second state, with the section taken along the longitudinal centerline of the sterile drape.

FIG. 2C is a cross-section the sterile drape assembly of FIG. 1C installed on a manipulator in the third state, with the section taken along the longitudinal centerline of the sterile drape.

FIG. 2D is a cross-section the sterile drape assembly of FIG. 1E installed on the manipulator in the fourth state, with the section taken along the longitudinal centerline of the sterile drape.

FIG. 3 is a schematic side view of an openable end portion of another embodiment of a sterile drape assembly having a press seal closure mechanism.

FIG. 4 is a schematic side view of an openable end portion of yet another embodiment of a sterile drape assembly having a magnetic closure mechanism.

FIG. 5 is a schematic plan view of an openable end portion of yet another embodiment of a sterile drape assembly having a perforated closure mechanism.

FIG. 6 is a schematic plan view of an openable end portion of yet another embodiment of a sterile drape assembly having a string-tear closure mechanism.

FIG. 7 is a schematic plan view of an openable end portion of yet another embodiment of a sterile drape assembly having a rod and interlocking tab closure mechanism.

FIG. 8A is a schematic diagram of a worksite with a manipulator in an undraped state.

FIG. 8B is a schematic diagram of the worksite of FIG. 8A with the manipulator partially draped by the sterile drape assembly of FIG. 1A in the first state.

FIG. 8C is a schematic diagram of the worksite of FIG. 8A with the partially draped manipulator in a stowed state outside of a sterile filed.

FIG. 8D is a schematic diagram of the worksite of FIG. 8A with the partially draped manipulator having been extended out of the stowed stated but still outside of the sterile field.

FIG. 8E is a schematic diagram of the worksite of FIG. 8A with the manipulator partially draped by the sterile drape assembly of FIG. 1A in the third state.

FIG. 8F is a schematic diagram of the worksite of FIG. 8A with the manipulator draped by the sterile drape assembly of FIG. 1A in the fourth state.

FIG. 8G is a schematic diagram of the worksite of FIG. 8A with the manipulator draped by the sterile drape assembly of FIG. 1A in the fourth state having been moved into the sterile field.

FIG. 8H is a schematic diagram of the worksite of FIG. 8A with an instrument sterile adaptor that is coupled to the sterile drape assembly prior to the sterile drape assembly being mounted on the manipulator.

FIG. 8I is a schematic diagram of the worksite of FIG. 8A with an instrument sterile adaptor mounted on the manipulator and sterile drape.

FIG. 8J is a schematic diagram of the worksite of FIG. 8A with an instrument sterile adaptor being mounted on the manipulator through the closure mechanism 150.

FIG. 8K is a schematic diagram of the worksite of FIG. 8A with an instrument sterile adaptor mounted on the manipulator without a sterile drape assembly present.

FIG. 8L is a schematic diagram of the worksite of FIG. 8A with an instrument mounted on the instrument sterile adaptor.

FIG. 8M is a schematic diagram of another worksite with a manipulator partially draped by the sterile drape assembly of FIG. 1A in the first state.

FIG. 8N is a schematic diagram of the worksite of FIG. 8M with the manipulator fully draped by the sterile drape assembly of FIG. 1A in the fourth state.

FIG. 8O is a schematic diagram of the worksite of FIG. 8M with the manipulator moved into a sterile field.

FIG. 9 is a schematic plan view of another embodiment of a sterile drape assembly having a hold-open device, shown with the sterile drape assembly in the third state.

FIG. 10A is a cross-section of the sterile drape assembly of FIG. 9 in the second state, with the section being taken along a longitudinal centerline of the sterile drape.

FIG. 10B is a cross-section of the sterile drape assembly of FIG. 9 in the third state, with the section being taken along a longitudinal centerline of the sterile drape.

FIG. 10C is a cross-section of the sterile drape assembly of FIG. 9 in the third state, with the section being taken along a longitudinal centerline of the sterile drape.

FIG. 11A is a cross-section of another embodiment of a sterile drape assembly having a wiper device, with the sterile drape assembly in the second state and the section being taken along a longitudinal centerline of the sterile drape.

FIG. 11B is a cross-section of the sterile drape assembly of FIG. 11A in the third state, with the section being taken along a longitudinal centerline of the sterile drape.

FIG. 11C is a cross-section of the sterile drape assembly of FIG. 11A in the third state, with the section being taken along a longitudinal centerline of the sterile drape.

FIG. 12A is a cross-section of another embodiment of a sterile drape assembly having pleats, with the section being taken along a longitudinal centerline of the sterile drape.

FIG. 12B is a cross-section of the sterile drape assembly of FIG. 12A, with the section being taken along a longitudinal centerline of the sterile drape.

FIG. 12C is a cross-section of the sterile drape assembly of FIG. 12A, with the section being taken along a longitudinal centerline of the sterile drape.

FIG. 13A is a schematic perspective view of another embodiment of a sterile drape assembly having a draping assist handles.

FIG. 13B is a schematic perspective view the sterile drape assembly of FIG. 13A being installed on a manipulator.

FIG. 14A is a schematic perspective view of another embodiment of a sterile drape assembly having a drape installation indicator.

FIG. 14B is a schematic perspective view the sterile drape assembly of FIG. 14A in the fourth state installed state on a manipulator.

FIG. 15 is a schematic perspective view another embodiment of a sterile drape assembly having an instrument sterile adaptor.

FIG. 16 is a schematic perspective view of another embodiment of a sterile drape assembly having hole for coupling with an instrument sterile adaptor.

FIG. 17A is a schematic perspective view the sterile drape assembly of FIG. 1A.

FIG. 17B is a schematic perspective view the sterile drape assembly of FIG. 17A with a second end folded partially into a remainder of the sterile drape assembly.

FIG. 17C is a schematic perspective view the sterile drape assembly of FIG. 17A with the second end folded further into a remainder of the sterile drape assembly.

FIG. 17D is a schematic perspective view the sterile drape assembly of FIG. 17A in a partially inverted state.

FIG. 18A is a schematic perspective view the sterile drape assembly of FIG. 1A.

FIG. 18B is a schematic perspective view the sterile drape assembly of FIG. 18A with a first end folded partially over a remainder of the sterile drape assembly.

FIG. 18C is a schematic perspective view the sterile drape assembly of FIG. 18A with the first end folded further over a remainder of the sterile drape assembly.

FIG. 18D is a schematic perspective view the sterile drape assembly of FIG. 18A in a partially inverted state.

### DETAILED DESCRIPTION

As noted above, it can be challenging to appropriately drape equipment, such as for example, a manipulator of a computer-assisted medical system, used in an environment in which a sterile field is desirable without contaminating the sterile field and without interfering with preparation of the worksite. The following description will refer primarily to manipulators as an example of equipment to be draped, but it should be understood that the disclosure is applicable to any other equipment for which such maintenance of a sterile field may be desirable, such as, for example, Mayo stands, endoscopes, microscopes, C-arms, etc.

In some systems, equipment for use during a procedure can be moved away from a worksite prior to the procedure and can be draped while at a distance from the worksite. This may make it easier to drape the equipment while in parallel preparations of the worksite for the procedure are being performed, as there may be more room to maneuver without interference both around the equipment and at the worksite. For example, in the case of a manipulator system being deployed in a surgical context, a manipulator may be moved a distance away from an operating table to facilitate draping of the manipulators by technicians at a location where there is plenty of room to maneuver without interference, while in parallel the worksite is being prepared for the procedure (e.g., a patient is being prepared on the operating table). In some cases, the equipment remains within the sterile field while being draped away from the worksite, so that it can be returned to the worksite after draping without contaminating the sterile field.

However, in other systems, it might not be possible for the equipment to be easily moved away from the worksite to gain the needed room to drape the equipment while still also keeping the drape sterile. For example, in some surgical manipulator systems, the manipulator may be attached to a portion of the operating table, thus making it difficult to move the manipulator away from the operating table while remaining in the sterile field. Thus, if it is attempted to drape such equipment in parallel with preparations of the worksite, because the equipment remains in or near the worksite as the space around the worksite may be occupied by various persons and/or devices associated with preparation of the worksite, which can get in the way of and interfere with the draping of the manipulator, and conversely the manipulator and/or the technician attempting to drape it can get in the way of and interfere with the people and/or devices associated with preparation of the worksite. For example, in a surgical context preparing the worksite may include things such as getting a patient situated on an operating table, draping the patient, preparing an entry site (e.g., incision), installing medical equipment to the patient, or otherwise preparing the patient or medical equipment for the procedure. Thus, the patient, the staff attending the patient, and the medical equipment may occupy much of the space around the worksite, and these people and devices may get in the way of and interfere with the manipulator and an individual attempting to drape it, and vice versa. Thus, draping the manipulator in parallel with preparation of the worksite can be challenging.

In some systems with equipment that cannot easily be moved from the worksite, the above challenges may be partially addressed by stowing the equipment near the worksite but at a location in which it will not interfere with preparation for the procedure, such as under an operating table in a surgical context. The stowed equipment may then be returned to its regular position and draped after the other preparations are completed. This may reduce the extent to which the draping of the equipment interferes with the preparing of the worksite. However, even after the preparations of the worksite are completed, the objects in the prepared worksite may continue to make draping of the equipment difficult. For example, in the surgical context, once the worksite is prepared the patient and at least some of the medical equipment and staff attending to the patient remain present in the worksite, and thus these people and equipment may continue to interfere with installation of the drape, and vice versa. Furthermore, in some circumstances, it may be desirable to decrease an amount of time between completion of the preparation of the worksite (e.g., preparing a patient) and the beginning of the operation, and attempting to install a sterile drape after completion of preparation of the worksite may delay the beginning of the medical procedure. Thus, attempting to stow equipment in the worksite during the preparation of a worksite and then draping the equipment after preparation of the worksite can be challenging.

In view of the challenges associated with draping the equipment in parallel with or after preparation of the worksite, one might consider attempting to install the drape prior to preparation of the worksite. However, in some circumstances, draping the manipulator before the worksite is prepared can risk jeopardizing the sterility of the sterile field. In particular, as noted above, in some systems in which the equipment cannot easily be moved away from the worksite while also remaining in the sterile filed, such as a system with a manipulator attached to an operating table, to facilitate the preparation of the worksite the equipment may be stowed at a location that is in or near the worksite, such as below an operating table. However, such stowed positions can be outside of the region that will ultimately be the sterile field. Thus, in such a system, if the equipment were to be draped prior to preparation of the worksite, then when the equipment is later stowed during preparation of the worksite, the sterile drape mounted on the manipulator will leave the sterile field and enter a non-sterile environment. This movement of the sterile drape outside of the sterile field compromises the sterility of the exterior surfaces of the drape by potentially exposing those surfaces to contaminants, and thus when the manipulator and drape end up being returned to the sterile field after preparation of the worksite has been complete, the compromised surfaces of the drape may compromise the sterility of the sterile field. (Note that moving the drape outside of the sterile field does not necessarily result in the drape being actually contaminated, but because the continued sterility of the drape cannot be guaranteed once it has been moved outside of the sterile field, under some medical guidelines and best practices the drape would be presumed to have been contaminated, and therefore the sterile field also regarded as compromised). Also, the sterile exterior of the drape may be contaminated by inadvertent contact as people work around the draped equipment while preparing the worksite. Thus, even if the stowed position of the equipment is within the sterile field, the sterility of the drape may nevertheless not be guaranteed due to the possibility of inadvertent contamination due to the proximity of the stowed position to the worksite. Accordingly, attempting to drape the equipment prior to preparation of the worksite can also pose its own challenged, under some circumstances.

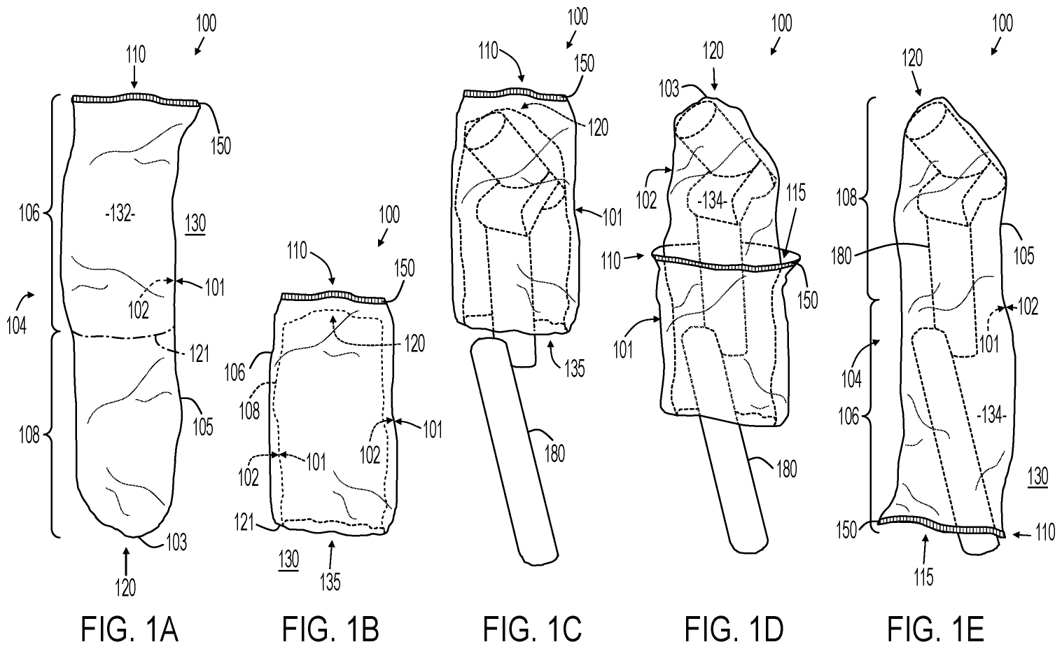

Accordingly, to address these and other challenges, various embodiments disclosed herein comprise a sterile drape that can be installed on equipment (e.g., a manipulator) before preparation of a worksite, be subsequently stowed away with the equipment outside of a sterile field during preparation of the worksite, and then be converted to a sterile condition just before or upon being returned to the sterile field once preparation of the worksite is completed. In some embodiments, the sterile drape comprises a drape body made of a draping material, such as a sheet of flexible plastic or polymer material for example, which is configured as an elongated hollow body comprising two opposite ends coupled together by a side wall (or side walls) extending along the length of the drape body and surrounding and defining an interior space within the drape body. A first end of the drape is closed, for example by integrally extending the draping material from, or coupling a separate piece of draping material (which may be the same or a different material as the side walls) to, the side wall(s) so as to cover and close one end of the hollow interior space. The second end of the drape is releasably closed or closable by a closure mechanism that has a closed state and an open state. (See, for example, the drape assembly 100 illustrated in FIG. 1A.) With the closure mechanism in the closed state, the interior space defined by the drape body is fully enclosed, while the closure mechanism in the open state provides an opening between the interior space and an exterior environment. The closure mechanism is configured to facilitate transition from the closed state to the open state. In some embodiments the closure mechanism may allow for reversable transitioning between the closed state and the open state, but in other embodiments the closure mechanism may only allow for a single transition from the closed state to the open state.

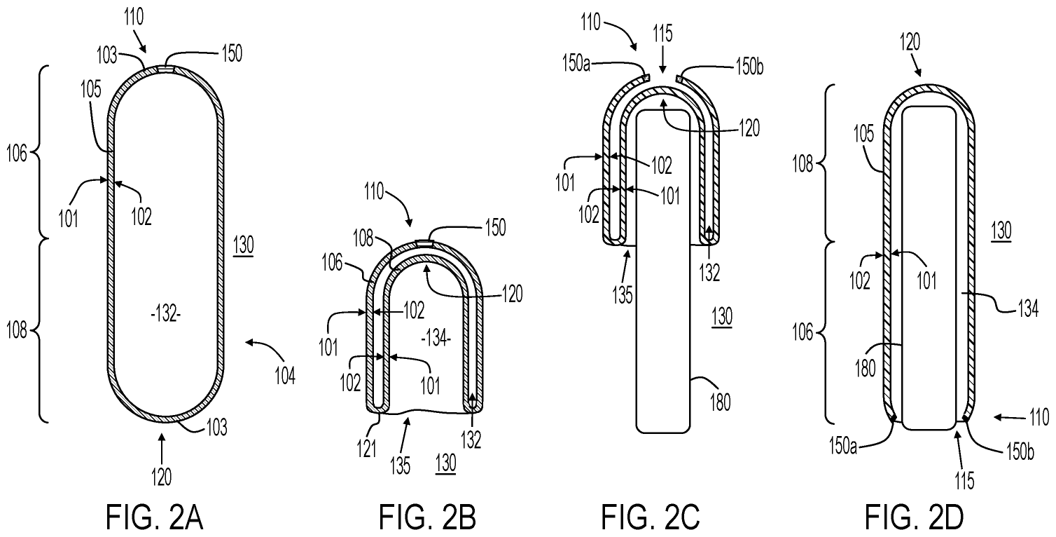

In some embodiments, the sterile drape has a partially inverted state, in which a portion of the drape body is folded back over or into another portion of the drape body (see, e.g., FIGS. 1B and 2B). In the partially inverted state, a portion of a first surface of the drape body is exposed to an exterior environment while a second surface of the drape body, opposite from the first surface, is exposed to an internal sterile environment within an enclosed interior region of the drape. Moreover, in the partially inverted state, the drape defines a partially enclosed space with an opening at one end thereof in which equipment may be received. The drape may be installed on equipment (e.g., a manipulator) in this partially inverted state, such that a portion of the equipment is received within the partially enclosed space defined by the drape. Sterile adapters can be attached as needed. After the equipment is partially draped with the partially inverted surgical drape, the equipment and the drape may be stowed outside of the sterile field. When thus stowed, the sterility of the exposed portion of the first surface of the drape is compromised by exposure to the non-sterile environment, but the unexposed, second surface remains sterile because it is within the sterile interior volume within the drape while stowed. Immediately before being returned into the sterile field (or in some embodiments upon being returned), the sterile drape may be converted into a fully inverted state (also referred to herein as a “second state”) in which the second surface of the drape faces outwardly and is exposed to the exterior environment, which is now in a sterile field, while the portion of the first surface previously exposed to the non-sterile field now faces inwardly into the partially enclosed space in which the equipment is received. In other words, in the fully inverted state, what was previously an exposed exterior portion of the first surface now is an interior surface of the drape, and the second surface, previously enclosed within the inverted drape volume is now an exterior surface of the drape.

To convert the sterile drape from the partially inverted state to the fully inverted state, in some embodiments one end of the sterile drape is opened by opening a closure mechanism, which is described in greater detail below. The now-opened end may then be moved along a length of the equipment to unfold the drape and cover a remaining portion of the equipment that was not covered in the partially inverted state. As the opened end is moved along the length of the equipment, eventually the drape will become fully unfolded and inverted such that the second surface, which was in the internal environment enclosed inside the drape, now faces an external environment.

Once the drape has been converted to the fully inverted state, all of the external exposed surface portions of the drape are sterile, and the compromised surface portion of the drape faces an internal space bound by the drape and are not directly exposed to the sterile field. Thus, in the fully inverted state the drape does not compromise the sterility of the sterile field.

Moreover, because the initial installation of the drape, including sterile adapters and other attachments in some embodiments, on the equipment can occur prior to preparation of the worksite, interference between the installation of the drape and the preparation of the worksite can be avoided during at least this phase of the drape installation. Thus, the amount of interference that occurs due to installation of the sterile drapes is reduced in various embodiments disclosed herein as compared to installation of conventional sterile drapes during or after preparation of the worksite, and the installation process of the sterile drapes disclosed herein can therefore be easier than the installation process of conventional sterile drapes.

As noted above, interference between the installation of the drape and the preparation of the worksite can be avoided during the initial installation of the drape, but some interference may occur during the subsequent process of converting the drape to the fully inverted state, which happens after preparation of the worksite. However, the process of initially fitting a sterile drape (whether a conventional drape or one of the embodiments disclosed herein) onto the equipment can be a relatively more difficult and slower part of the overall installation process than the process of converting the drapes disclosed herein from the partially inverted state to the fully inverted state. The initial fitting of a sterile adaptor to equipment may include relatively more difficult and/or time consuming operations such as aligning features of the drape with corresponding target locations of the equipment and/or installing an instrument sterile adaptor (which may be part of or fitted over the drape) onto the equipment. Therefore, although some interference may occur while converting the sterile drape to the fully inverted state in accordance with various embodiments disclosed herein, it occurs during a relatively easy and brief part of the installation process and thus is less disruptive and impactful. Accordingly, the overall degree of interference and difficulty associated with installing various embodiments of drapes disclosed herein is relatively minimal.

Various embodiments of the disclosure will be described in greater detail below, with reference to the figures. In the figures, underlined reference numbers disposed on an illustrated surface are intended to indicate the surface upon which they are disposed (see, for example, the reference number 101 in FIG. 1A). A lead line with an arrow that touches a surface may also be used to indicate the surface touched by the arrow, from the perspective indicated by the arrow (see, for example, the reference number 101 in FIG. 1D). A dashed lead line with an arrow touching a surface may be used to indicate a surface that is obscured in the figure (see, for example, the reference number 102 in FIG. 1A). A lead line with an arrow that is spaced apart from and not touching a body may indicate a general region, portion, part, or the like, that the arrow points towards (see, for example, the reference number 110 in FIG. 1A). A lead line without an arrow may indicate a structure or other element touched by the lead line (see, for example, the reference number 150 in FIG. 1A). Reference numbers enclosed by dashes refer to a volume or environment within which the reference number is disposed (see, for example, the reference number 132 in FIG. 2A). A lead line with an arrow may also be used to indicate volume or environment at or into which the arrow points (see, for example, the reference number 132 in FIG. 2B). In cases in which an interior volume or environment is obscured in a figure by a body defining the volume/environment, the interior volume or environment may be indicated by a reference number enclosed in dashes that is disposed on an exterior (visible) surface of a structure that (at least partially) defines the volume (see, for example, the reference number 132 in FIG. 1A), or by a dash-lined arrow that points at a location that corresponds generally to the volume.

### I. Sterile Drape Assembly

FIGS. 1A-2D illustrate an embodiment of a sterile drape assembly 100 (“drape assembly 100”). FIGS. 1A-1E comprise perspective views illustrating the drape assembly 100 in various states. FIGS. 2A-2D comprise schematic cross-sectional views of the drape assembly 100 in various states.

As shown in FIGS. 1A and 2A, the drape assembly 100 comprises a drape body 104 and a closure mechanism 150 coupled to the drape body 104. The drape body 104 comprises a side wall 105 (also referred to herein as lateral wall) extending along a length of the drape assembly 100 between two opposite ends, namely a first end 110 and second end 120. As shown in FIG. 2A, the side wall 105 of the drape body 104 has a first surface 101 and a second surface 102 opposite from the first surface 101. In various embodiments, side wall 105 has a generally annular cross-section taken in a transverse plane to the longitudinal axis of the drape body 104. The side wall 105 surrounds and (at least partially) defines a hollow interior volume around a longitudinal axis of the drape body 104 in a fully extended state along a longitudinal dimension thereof. (Note that “annular” as used here does not require a circular, or even rounded, shape, but rather refers generally to a shape with a relatively thin rim that encloses an open interior area inside the rim.) In other words, the drape body 104 when fully extended longitudinally (see in FIGS. 1A, 1E, 2A, and 2D) forms an elongated hollow body, which may be generally tubular in shape. (Note that “tubular” as used here can include but does not require a circular, or even round, cross-section. A cross-section of the drape body 104 may have any shape, and the cross-section need to not be uniform along the length of the body; tapered shapes are contemplated in various embodiments.) In the description below, it is assumed for convenience and ease of understanding that one side wall 105 is present, and this single side wall 105 loops continuously around the longitudinal axis to form the hollow elongated shape of the drape body 104, but it should be understood that in other embodiments multiple distinct side walls 105 could be joined together to provide an integral structure forming the same or similar shape. The term side wall 105 is intended to be inclusive in scope of both embodiments.

As noted above, the drape body 104 comprises a first end 110 and a second end 120 between which the side wall 105 extends. The drape body 104 may comprise an end wall 103 at one or both of the ends 110 and 120. These end walls 103 may be integrally coupled with (i.e., part of the same monolithic body as) the side wall 105 as shown in FIGS. 1A-2D, or they may be separate pieces that are joined to the side wall 105 to form an integral structure therewith. In some embodiments, the side wall 105 may smoothly transition into the ends 110 and 120 such that it may not be possible to distinguish end walls 103 from the side wall 105, in which case the end walls 103 may be regarded as being another part of the side wall 105. A closure mechanism 150, described in greater detail below, may be disposed in or coupled to an end wall 103 at the first end 110, or in some embodiments the closure mechanism 150 itself may serve as and take the place of an end wall 103.

As noted above, the side wall 105 surrounds and defines at least one interior volume. More specifically, the side wall 105, the end walls 103, and the closure mechanism 150 cooperate to surround and define the at least one interior volume. The interior volume(s) may be fully or partially enclosed by the side wall 105, end wall(s) 103, and closure mechanism 150, depending on a state of the drape assembly 100 and whether the closure mechanism 150 is open or closed. In addition, in some states there are multiple distinct interior volumes defined by the drape assembly 100, as will be further explained below. The number and configurations of the interior volumes defined by the drape assembly 100 may vary depending on the state of the drape assembly 100, as described below. The term “volume” as used herein is not intended to refer to a specific amount of space (e.g., a particular volumetric volume) or express a constant quantity, but rather is used herein to denote an enclosed or partially enclosed space, the size of which can change depending on the state of the drape assembly 100.

More specifically, in the embodiment illustrated in FIGS. 1A-2D, the second end 120 is permanently closed by an end wall 103, which is integrally coupled with the side wall 105. The first end 110, on the other hand, is not permanently closed, but rather is releasably closed by a closure mechanism 150 disposed at the first end 110, which is described in greater detail below. Thus, whether the interior volume is fully or partially enclosed may depend on that state of the closure mechanism 150 being open or closed.

In an initial state (see FIGS. 1A and 2A), an interior volume defined by the drape body 104 comprises the interior volume 132, and this interior volume 132 is fully enclosed (assuming that the closure mechanism 150 is closed). In a fully inverted state (described below) (see FIGS. 1E and 2D), an interior volume defined by the drape body 104 comprises the interior volume 134, which is partially enclosed (with the closure mechanism 150 opened) and configured to receive draped equipment, such as a manipulator 180. The interior volume 134 is not yet present in the drape assembly 100 when in the initial state, and in the fully inverted state the interior volume 132 is no longer present due to the drape assembly 100 having been inverted. Further, as is apparent from the FIGS. 2A-2D, a second surface 102 of the drape body 104 faces (forms the interior surface surrounding) the interior volume 132 and a first surface 101 of the drape body 104, opposite the second surface 102, faces (forms the interior surface surrounding) the interior volume 134. In a partially inverted state (described below) (see FIGS. 1B and 2B), both the interior volume 132 and the interior volume 134 are present in the drape assembly 100. More specifically, the drape body 104 is in a state in which the interior volume 132, defined by the interior surface 102, surrounds the interior volume 134, defined by the interior surface 101, as shown in FIG. 2B. The aforementioned states of the drape assembly 100 are described in greater detail below. Thus, in the various states of the drape assembly 100, the first surface 101 and the second surface 102 alternate which surface, or portions thereof, faces an exterior environment 130 and which faces into the interior volume 132 or 134 within the drape assembly 100, as described below.

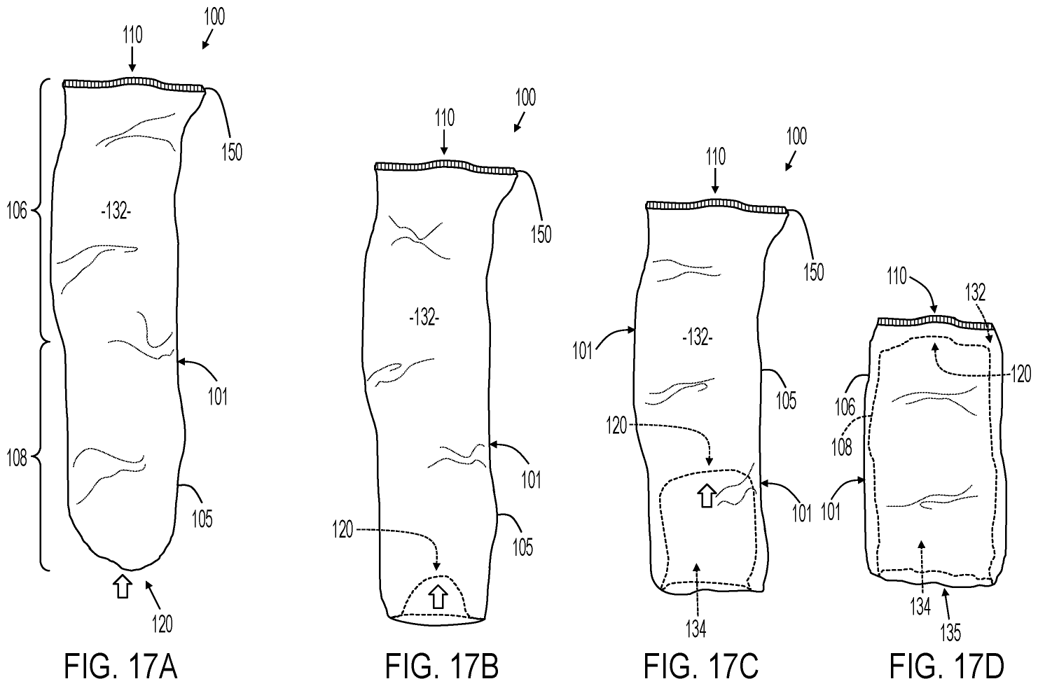

In other embodiments, the second end 120 may be permanently closed by a separate piece of material coupled to the side wall 105, rather than by material integrally coupled with the side wall 105. In still other embodiments, the second end 120 of the drape may be closed in some states and open in other states, rather than being permanently closed. For example, the second end 120 may be releasably closed by another closure mechanism (not illustrated), or the second end 120 may comprise a hole that is open in some states but ultimately closed when the drape is fully installed, as described below with reference to FIG. 17.

As mentioned above, a closure mechanism 150 is coupled to (or is part of) the drape body 104 at the first end 110 (see FIGS. 1A and 2A). The closure mechanism 150 is releasably closed or closable, and when closed the closure mechanism 150 closes the first end 110 of the drape assembly 100. Specifically, in the state illustrated in FIGS. 1A and 2A, the closure mechanism 150 is in a closed state, or is capable of being placed in the closed state, and the closure mechanism 150 is configured to facilitate transitioning from the closed state to the open state. In the closed state of the closure mechanism 150, the first end 110 of the drape assembly 100 is closed by the closure mechanism 150 such that communication of contaminants between an interior volume 132 and the external environment through the first end 110 is substantially prevented or inhibited. The closed state may comprise, but does not necessarily have to comprise, an airtight seal or a liquid tight seal. Regardless of the seal that may be formed, in various embodiments, the closed state is sufficient to provide a barrier between the interior volume 132 and the environment outside the interior volume 132 sufficient to maintain sterility of the interior volume 132. In the open state of the closure mechanism 150, the first end 110 of the drape assembly 100 is opened to provide an opening 115. The transitioning of the closure mechanism from the closed state to the open state may be referred to herein interchangeably as “releasing” the closure mechanism or as “opening” the closure mechanism. The closed state may comprise, but does not necessarily have to comprise, an airtight seal or a liquid tight seal.

As noted above, in some embodiments, the first end 110 may also comprise an end wall 103, which may be coupled to or integrally coupled with (i.e., part of the same monolithic body as) the side wall 105, with the closure mechanism 150 being disposed in a portion of the end wall 103 so as to form an opening in the end wall 103 in an open state of the closure mechanism 150. In other embodiments, the closure mechanism 150 may act as the end wall 103, and may be coupled directly to the side wall 105. As noted above, distinctions between end walls and side walls are made herein to aid understanding, but it should be understood that in practice the two may continuously run into one another and there may be no principled way to determine where an end wall begins and a side wall ends.

The walls of the drape body 104, including the side wall 105, may be made from a draping material (or multiple layers of different draping materials), such as a sheet of flexible plastic, a sheet of polymer, a woven material, or other material that is suitable for draping equipment to establish a sterile barrier. Suitable materials may include, for example, materials that are flexible enough to facilitate installation of the drape on the equipment and to allow for articulation of the draped equipment in embodiments in which the draped equipment is articulable. In some implementations, the drape material (or one or more of the drape material layers in a multi-layered drape body 104) may also be liquid tight to prevent the transfer of fluids through the drape body 104. In some implementations, the drape material (or one or more drape material layers) may be non-absorbent. Examples of flexible, liquid tight and non-absorbent materials include flexible plastics and sheets of polymer. In some implementations, some portions of the drape body 104 (or in some cases, all of the drape body 104) may comprise an absorbent material. In some implementations, the drape body 104 comprises a combination of one or more absorbent outer layers and one or more non-absorbent liquid tight inner layers. In some implementations, the drape material may be non-porous as to liquids but may be porous as to gases-ePTFE (Gore-Tex) is one example of such a material. In some implementations, porosity or openings (e.g., valves) may be artificially provided in an otherwise non-porous drape material, for example to bring air from outside the drape to the interior for convective cooling of the enclosed equipment. In some embodiments, the draping material for the drape body 104 may also advantageously have any combination of the following properties: resistance to tears and punctures so as to maintain the sterile barrier in case the draped equipment contacts other equipment or personnel; lint free; does not form dust; fire retardant; non-toxic; and sterilizable. Also, in some embodiments the drape material is transparent so operators can view the draped equipment more easily and install adapters or other attachments more readily. Some non-limiting examples of suitable drape materials include non-woven fabrics such as a polyethylene and polyester blend non-woven fabric; films or membranes such as a polyethylene, Polyurethane, or ePTFE (Gore-Tex); and woven fabrics such as cotton or polyester fabrics. Some additional examples of suitable drape materials, which may be used as films or as fibers in woven or non-woven fabrics, include Thermoplastic polyurethane (TPU), Polyether polyurethane, Polyester polyurethane, and Linear Low-Density Polyethylene (LLDPE). In various embodiments, the drape body 104, at least at the side wall 105, may be made of a transparent material to aid in observation of placement of the drape assembly 100 over equipment. Various markings may also be provided to aid in such placement and to aid in an understanding of orientations during placement and/or alignments with parts of the equipment to be draped.

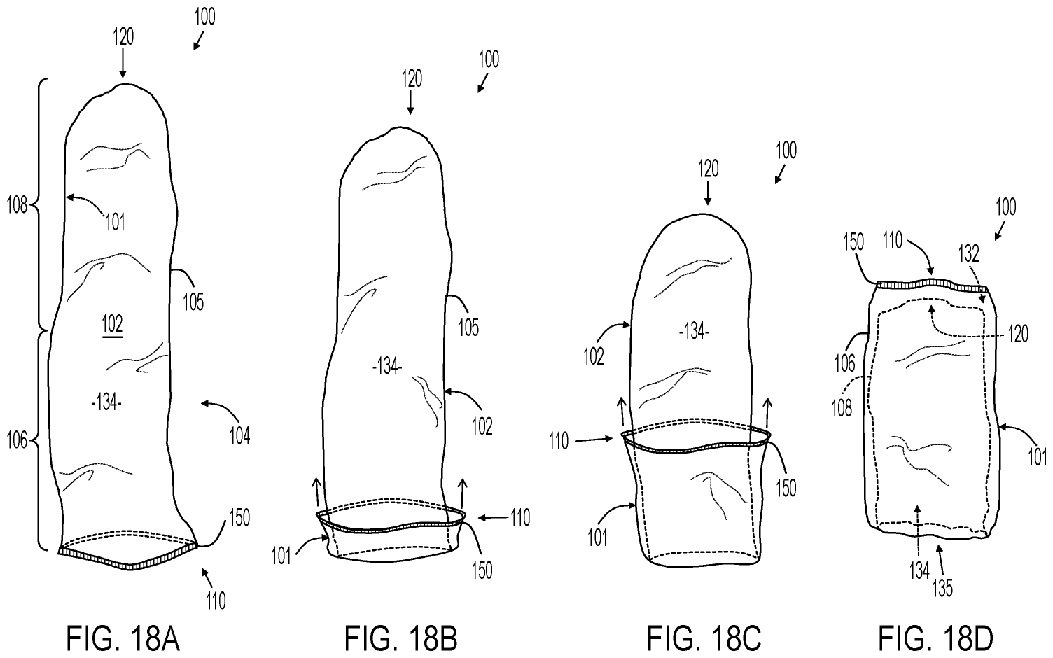

As noted above, the drape assembly 100 has an initial, fully extended state. The drape assembly 100 is in this state prior to being transitioned to a partially inverted, which is described below. As shown in FIGS. 1A and 2A, in the initial state the drape assembly 100 is fully extended along a longitudinal dimension of the drape assembly 100 such that two portions 106 and 108 of the drape assembly 100, corresponding roughly to two halves of the drape assembly 100, are disposed adjacent to one another sequentially along a longitudinal dimension of the drape. Note that the portions 106 and 108 are not necessarily equal in length. The two portions 106 and 108 are joined together at an intermediate portion 121, which is indicated in FIG. 1A with dash-dot lines. In the embodiment of FIGS. 1A and 2A, the first surface 101 of the drape body 104 faces an exterior environment 130 in the initial state, while the second surface 102 of the drape body 104 faces into an interior volume 132 in the initial state. However, as described in greater detail below and as those of ordinary skill in the art would understand from the present disclosure with reference to FIGS. 18A-18D, it is possible for the drape assembly 100 to have an initial state that is inverted relative to the state shown in FIG. 1A, with the second surface 102 facing the exterior environment 130 and the first surface 101 facing the interior volume 132. For purposes of the following description, it will be assumed that the initial state corresponds to that shown in FIGS. 1A and 2A, unless noted otherwise, as the subsequent states of the drape are not affected by which initial state of the drape is used.

As shown in FIGS. 1B, 1C, and 2B the drape assembly 100 can be reconfigured from the initial state into a partially inverted state, in which the portion 108 is received within and surrounded by (e.g., roughly concentrically by) the portion 106. In other words, in the partially inverted state of the drape assembly 100, the portion 106 overlaps and covers the portion 108, surrounding the perimeter of the portion 108 to enclose it. Thus, as shown in FIGS. 1B and 2B, in the inverted state the portions 106 and 108 are positioned at approximately the same position as one another along the longitudinal dimension of the drape assembly 100, in contrast to the initial state in which the portions 106 and 108 are positioned sequentially next to one another along the longitudinal dimension with the two portions 106 and 108 meeting at a location along the overall length (e.g., roughly in a midportion of the length) of the drape in the extended state and extending therefrom in opposite directions to each end 110 and 120, respectively.

As a result of this reconfiguration, the internal volume 134 mentioned above is formed within the portion 108, with the intermediate portion 121 at which the two portions 106 and 108 are joined together forming a rim of an opening 135 into the internal volume 134, as shown in FIGS. 1B and 2B. In the partially inverted state, the intermediate portion 121 also corresponds to a fold in the drape body 104 at which the drape body 104 is folded back over itself (or under itself, depending on perspective). In some embodiments, the intermediate portion 121 may be a portion of the side wall 105 that is continuous with (part of the same monolithic body as) and not visually or structurally discernable from the rest of the side wall 105, and thus the location of the intermediate portion 121 (and hence the boundary between the portions 106 and 108) depends on the location at which the drape body 104 is folded in the partially inverted state, which may vary from one drape assembly 100 to the next or from time to time within the same drape assembly 100. Thus, the precise location of the intermediate portion 121 might not always be apparent in the initial state, and it is indicated in FIG. 1A merely for the sake of understanding. However, in other embodiments the intermediate portion 121 may be a predetermined portion of the drape assembly 100 that is structurally different than the surrounding side wall 105, and which may thus be discernible even in the initial state. For example, the intermediate portion 121 may be a portion that is configured to be easier to fold, more resilient or resistant to tearing when folded, thicker, thinner, formed from a different material, or the like, relative to the surrounding portions of the side wall 105.

In the partially inverted state, the internal volume 132 is fully enclosed, and all parts of the second surface 102 face into the fully enclosed internal volume 132. In this state, the portions of the second surface 102 that are respectively parts of the portions 106 and 108 face one another, with the internal volume 132 defined therebetween. The portion of first surface 101 that is part of portion 106 of the drape body 104, on the other hand, faces outwardly and remains exposed to the exterior environment 106 in the partially inverted state, as it was in the extended state of the drape body 104 of FIGS. 1A and 2A. In contrast, as also shown in FIGS. 1B and 2B, in the partially inverted state, the portion of the first surface 101 that is part of portion 108 of the drape body 104 faces inwardly, creating and defining a partially enclosed interior volume 134 with an opening 135 communicably coupling the interior volume 134 with an exterior environment 130. As noted above, the size (volumetric volume) of the interior volumes 132 and 134 may vary, depending on the state of the drape assembly 100. As shown in FIG. 1C, in the partially inverted state, the interior volume 134 is configured to receive equipment to be draped by the drape assembly 100, such as the manipulator 180, for example. Specifically, a free end of the equipment may be inserted into the partially enclosed interior volume 134 via the opening 135.

As discussed in greater detail below with reference to the embodiment of FIGS. 17A-18D, the partially inverted state may be obtained by folding the portion 108 into the interior space defined by the portion 106, or by folding the portion 106 over the exterior of the portion 108, depending on how the drape assembly 100 is configured in the initial state of the drape assembly 100. Regardless of which initial state the drape assembly 100 is in or which method is used to reconfigure the drape assembly 100 into the partially inverted state, the partially inverted state has the same general configuration shown in FIGS. 1B and 2B.

The drape assembly 100 in the partially inverted state may also be referred to herein as a “partially inverted drape assembly 100.” Because the portion 108 is surrounded by the portion 106 in the partially inverted state, the portion 108 may be referred to herein as the inner portion 108, while the portion 106 may be referred to herein as the outer portion 106. Similarly, because the second surface 102 is wholly enclosed inside the sterile drape in the partially inverted state, the second surface is also referred to herein as the “inner surface 102.” It should be understood that references to “inner” and “outer” in conjunction with the portions 106 and 108 and with the surface 102 refer to their relative positions in the partially inverted state, as described above, but these references are not intended to imply anything about the positions of these parts in other states of the sterile drape assembly 100.

In some embodiments, the drape assembly 100 may be reconfigured from the initial state to the partially inverted state prior to being delivered to an end user (e.g., during a manufacturing process), while in other embodiments, the drape assembly 100 may be delivered to an end user in the initial state and the end user may reconfigure the drape assembly 100 into the partially inverted state. Methods of reconfiguring the drape assembly 100 into the partially inverted state are described in greater detail below with reference to FIGS. 17A-18D.

As shown in FIGS. 1D and 2C, the drape assembly 100 also has one or more transitional states in which the closure mechanism 150 has been released to open the first end 110 of the drape assembly 100, and in which the now-opened first end 110 has been or is being moved along a length of the partially draped equipment (e.g., manipulator 180) so as to transition the drape assembly 100 from the partially inverted state to a fully inverted state illustrated in FIGS. 1E and 2D. Upon release of the closure mechanism 150, the interior space 132 goes from being fully enclosed to being partially enclosed, with an opening 115. In addition, in the transitional states, the portions 106 and 108 at least partially overlap one another, with the amount of overlap progressively decreasing as the first end 110 is moved farther along the length of the equipment (or as the first end 110 moves along the drape to expose a greater length of the surface 102 to the exterior environment 130). In the partially inverted state, the partially enclosed interior volume 134 is bounded only by the portion of the surface 101 that is part of portion 108 of the drape body 104. However, as the drape assembly 100 is converted from the partially inverted state to the fully inverted state, the remaining portion of the surface 101, which faced away from the interior volume 134 in the partially inverted state (i.e., the portion of surface 101 that is part of the portion 106 of the drape body 104) begins to face inwardly and forms part of the boundary of the partially enclosed interior volume 134, thus progressively expanding the length of the partially enclosed interior volume 134. Moreover, the partially enclosed interior space 132 is progressively reduced in size as the first end 110 moves along the drape. The fully inverted state is reached once the first end 110 of the drape assembly 100 has been fully extended such that the portions 106 and 108 no longer overlap, as shown in FIGS. 1E and 2D. As shown in FIGS. 1E and 2D, in the fully inverted state, the surface 102 now faces externally and exposed to the exterior environment 130, whereas the surface 101 now faces inwardly into the partially enclosed interior volume 134 defined by the drape assembly 100 within which the equipment (e.g., manipulator 180) is received. The fully inverted state may also be referred to herein as simply the “inverted state.”

As is apparent from the above description and drawings, the interior volume 134 that is present in the partially inverted, transitional, and fully inverted states of the drape assembly 100 (states of FIGS. 1B-1E and 2B-2D) does not exist in the initial state of the drape assembly 100 of FIGS. 1A and 2A, but is created by virtue of reconfiguring the drape assembly 100 into the partially inverted state and then expanded while converting the drape assembly 100 to the fully inverted state. Conversely, the interior volume 132 ceases to exist at the fully inverted state (FIGS. 1E and 2D, but is present in the initial, partially inverted, and transitional states (states of FIGS. 1B-1D and 2B-2C).

### II. Closure Mechanisms

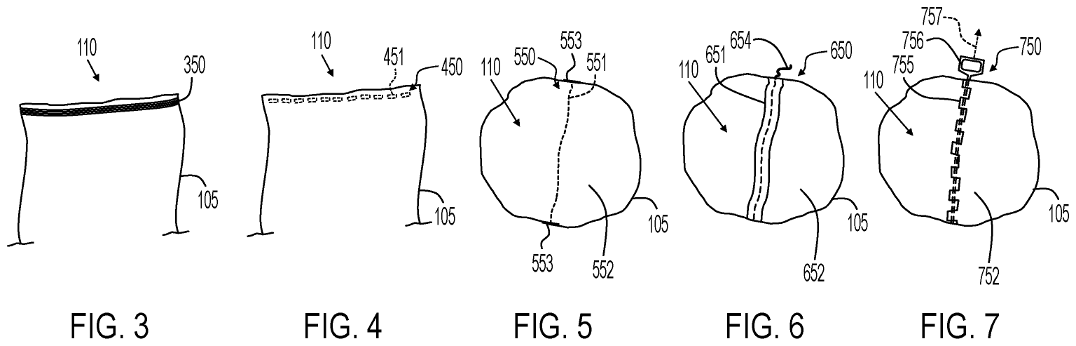

As described above, the closure mechanism 150 is configured to releasably close the first end 110. As used herein, “releasably” closing the first end 110 of the drape assembly 100 refers to closing the first end 110 in a manner that allows and facilitates subsequent opening thereof. In other words, the closure mechanism 150 is configured to place the first end 110 in a closed state while also allowing and facilitating a transition from the closed state to an open state. In other words, the closure mechanism 150 has one or more structural features that enable, assist, make easier, guide, control, and/or otherwise facilitate either the creation or uncovering/opening of an opening 115 in the first end 110. FIGS. 3-7 illustrate some non-limiting embodiments of closure mechanisms 350, 450, 550, 650, and 750 that can be used as the closure mechanism 150.

In some embodiments, the closure mechanism 150 releasably closes the first end 110 in a reversible manner, meaning that the closure mechanism can transition both from the closed state to the open state and from the open state to the closed state. For example, a press-seal closure mechanism 350 is illustrated in FIG. 3, which can be used as the closure mechanism 150. The press-seal closure mechanism 350 comprises two opposing strips having complementary flexible features formed on or coupled to the drape assembly 100 around a rim of an opening 115 (see FIG. 2C) in the first end 110 such that when the two strips are pressed together the complementary flexible features interlock and form a seal that closes the opening 115, and when the two strips are pulled apart the seal is released and the opening 115 is opened.

As another example, a magnetic closure mechanism 450 is illustrated in FIG. 4, which can be used as the closure mechanism 150. The magnetic closure mechanism 450 comprises one or more pairs of magnets 451 (or one or more pairs of a magnet and a ferromagnetic material) coupled to the drape assembly 100 on opposite sides of a rim of an opening 115 (see FIG. 2C) in the first end 110 such that when the pairs of magnets 451 are brought together they magnetically attract one another and thus hold together the opposite sides of the drape assembly 100 to which the magnets 451 are attached, thus closing the opening 115.

In addition, any other closure mechanism that can reversibly close the first end 110 may be used as the closure mechanism 150, such as, for example, a zipper, a tying closure (e.g., a drawstring, twist-tie, zip-tie, or the like tied around the opening 115), a buttoned closure, a snap closure, a closure comprising overlapping, interleaved, and/or folded flaps of drape material, a resealable adhesive strip, a hook-and-loop (e.g., Velcro) closure mechanism, etc.

In other embodiments, the closure mechanism 150 releasably closes the first end 110 in an irreversible manner, meaning that the closure mechanism 150 can transition from the closed state to the open state but cannot necessarily transition from the open state back to the closed state (or at least cannot do so easily or without substantially changing the mechanism). For example, FIG. 5 illustrates an embodiment of a tearable closure mechanism 550, which can be used as the closure mechanism 150. The tearable closure mechanism 550 comprises a wall 552 that covers and closes the first end 110 and one or more intentionally weakened regions 551 in the material forming the wall 552. The wall 552 may be integrally coupled to (i.e., part of the same monolithic body as) the side wall 105, or it may be a separate material that is coupled to the side wall 105, for example via adhesive, heat bonding (e.g., welding), mechanical attachment (e.g., stitching), or any other bonding technique. The intentionally weakened regions 551 may be formed in the material of wall 552 itself, for example as perforations through the wall 552, as regions of the wall 552 in which the material of the wall 552 is thinner, as regions of the wall 552 that have been weakened by application of a chemical or heat treatment, etc. Alternatively, the intentionally weakened regions 551 may be formed by joining a separate material to the wall 552 over an opening in the wall 552, with the separate material being weaker (easier to tear) than the surrounding material of the wall 552. The intentionally weakened regions 551 are configured to be torn apart to create an opening 115 in the wall 552, with the intentionally weakened regions 551 being easier to tear than the surrounding material of the wall 552 and/or side wall 105. This weakening of the material in the intentionally weakened regions 551 not only makes it easier to create the tear, but it also ensures (or at least increases the likelihood) that the tear will follow a predetermined path, corresponding to the pattern of the intentionally weakened regions 551, rather than extending in an unpredictable fashion. In some embodiments, the tearable closure mechanism 550 may also comprise reinforced regions 553, which are disposed adjacent to the intentionally weakened region 551. The reinforced regions 553 may be relatively stronger, more resilient, and/or tear resistant than other portions of the wall 552. The reinforced regions 553 may help to prevent the tear from extending beyond the intentionally weakened regions. In some embodiments, the reinforced regions 553 are disposed at specific locations of concern where unwanted tearing is expected, such as at opposite ends of the intentionally weakened region 551 as illustrated in FIG. 5. In other embodiments, the reinforced regions 553 may be provided all around (completely surrounding) the intentionally weakened region 551 to further reduce the risk of a tear propagating past the intentionally weakened region 551.

Another embodiment of an irreversible closure mechanism which may be used as the closure mechanism 150 is a string-tear closure mechanism 650, as illustrated in FIG. 6. The string-tear closure mechanism 650 may be similar to the tearable closure mechanism 550 in that it is configured to create an opening 115 in the first end 110 by tearing open the wall 652 that covers and closes the first end 110. However, unlike the tearable closure mechanism 550, the string-tear closure mechanism 650 is designed to facilitate the tearing open of the opening 115 by way of a string 654, wire, or other similar member. The string 654 (or similar member) may be embedded in or under a region 651 of the wall 652 (or of material joined to the wall 652) with a free end of the string 654 being accessible from outside the drape 600 and an opposite end of the string 654 being secured to the drape. Thus, when a user pulls the string 654 the string 654 cuts and/or tears through the wall along the region 651, thus forming the opening 115 in the first end 110. The region 651 may be intentionally weakened, similar to the intentionally weakened region 551. Alternatively, the region 651 may be of similar strength to the surrounding material, as the string 654 may be sufficient to allow for easy and controlled tearing even without weaking the region 651. Although not illustrated, reinforced regions may also be provided, similar to the reinforced regions 553.

As another embodiment of an irreversible closure mechanism, FIG. 7 illustrates an interlocking tab closure mechanism 750, which may be used as the closure mechanism 150. The interlocking tab closure mechanism 750 comprises a series of tabs 755 arranged around a rim of an opening 115 in the first end 110 such that, when opposing sides of the rim are brought together, the tabs 755 mesh or interlock together, as illustrated, thus closing the opening 115 in the interlocked state. A rod 756, such as a flexible rod made of a polymeric material, is extended through openings in each of the tabs 755 in the interlocked state, thus holding the tabs 755 in the interlocked state and holding the opening 115 closed. To open the interlocking tab closure mechanism 750, the rod 756 may be pulled in the direction indicated by the arrow 757, thus removing the rod 756 from each of the tabs 755. With the rod 756 removed, the tabs 755 are now free to leave the interlocked state, thus opening the opening 115. The rod 756 may have a handle at a free end thereof to facilitate pulling of the rod 756 out of the tabs 755. In some embodiments, the tabs 755 may be relatively thicker and/or stronger and/or more rigid than the surrounding material of the end wall 752 to prevent the rod 756 from inadvertently tearing through the tabs 755. Although not illustrated, reinforced regions may also be provided, similar to the reinforced regions 553.

Although it may be technically possible to reclose the closure mechanisms 550, 650, and/or 750, the closure mechanisms 550, 650, and 750 are considered as irreversible because they cannot readily be put back into their original closed states without significant and time-consuming repair/reconstruction efforts.

### III. Systems Utilizing Sterile Drape Assembly

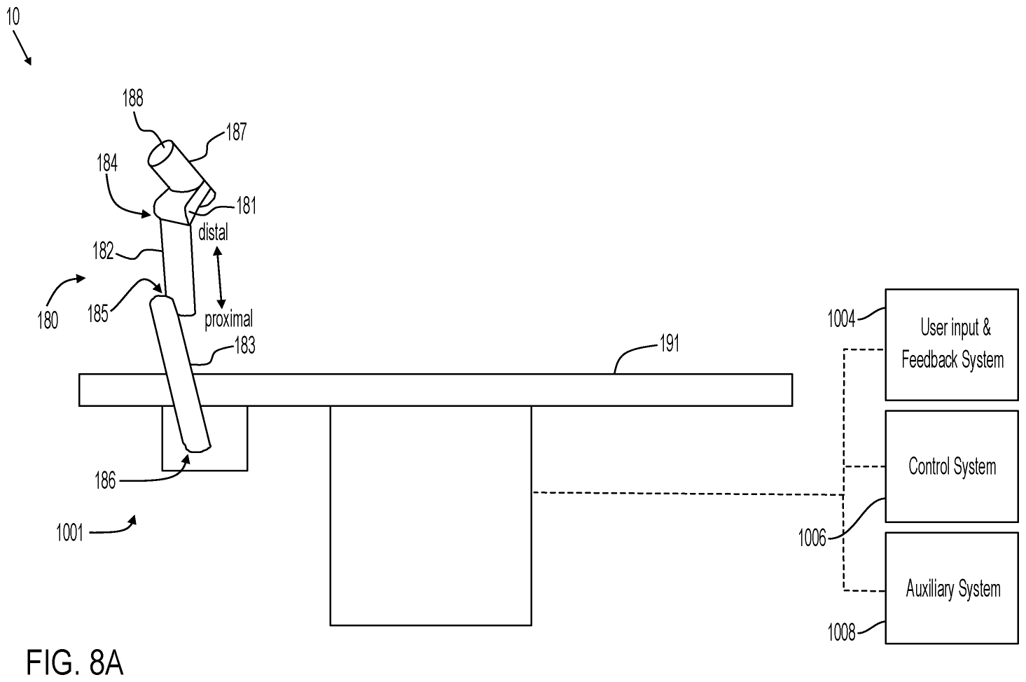

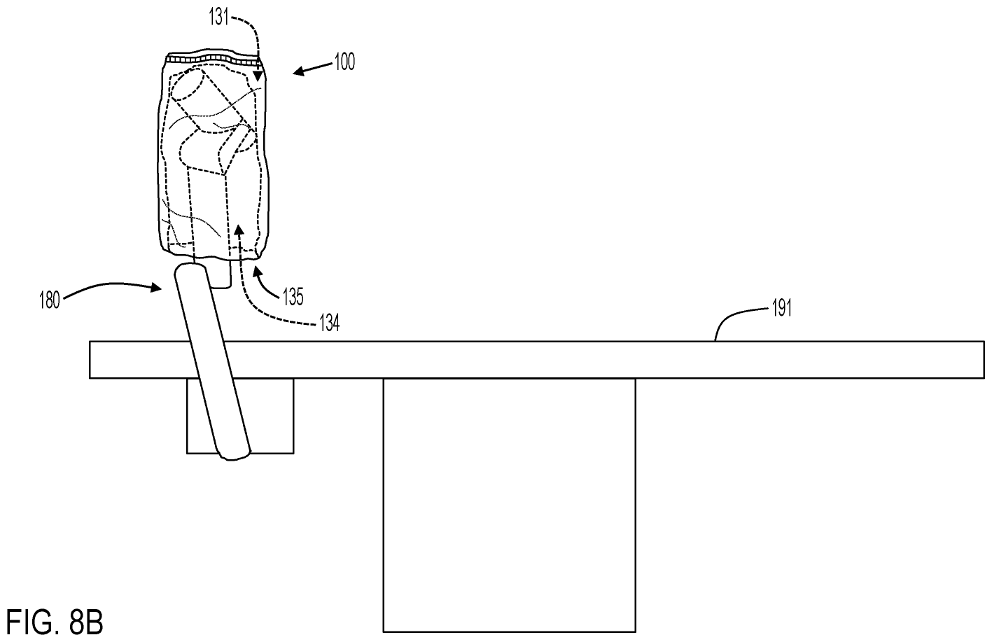

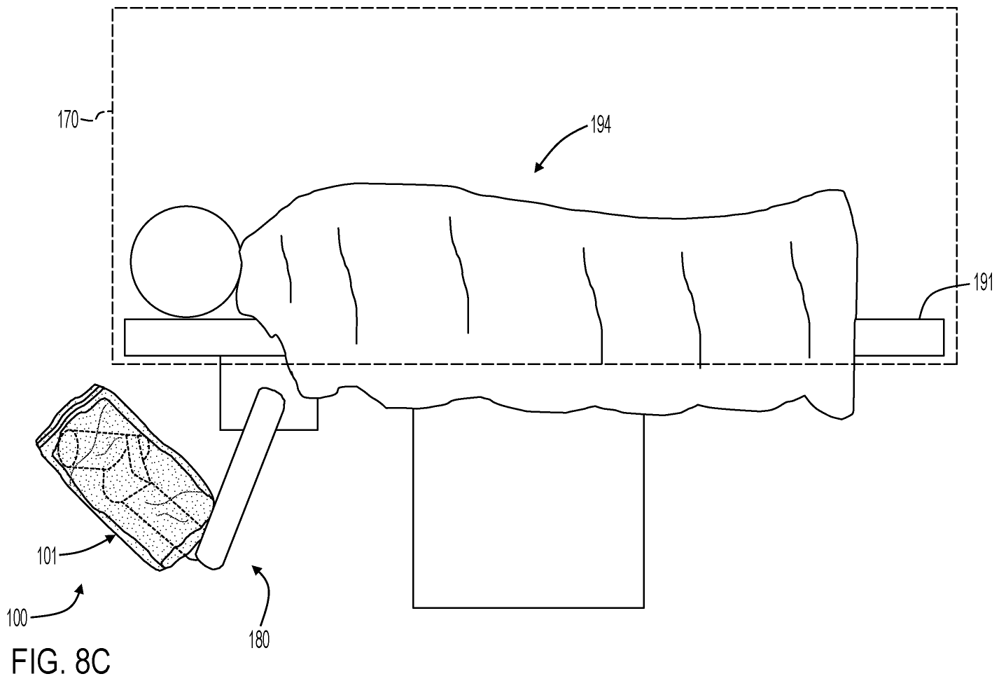

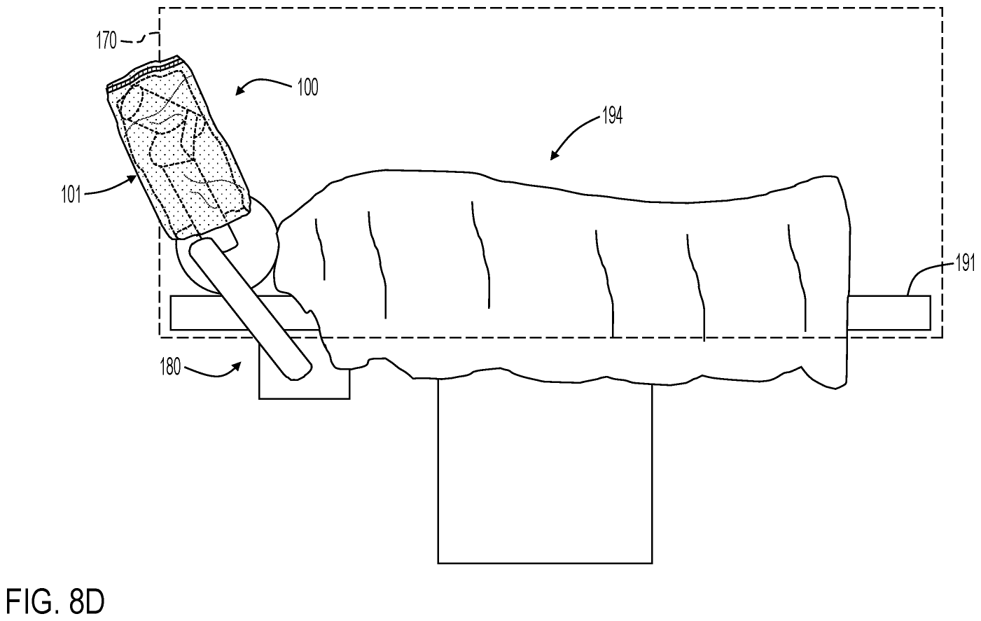

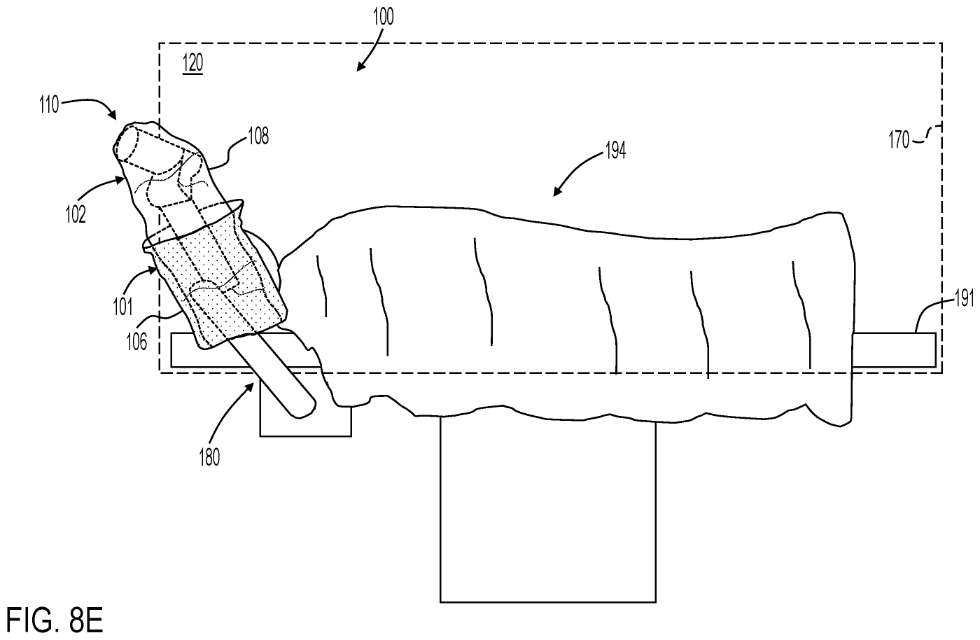

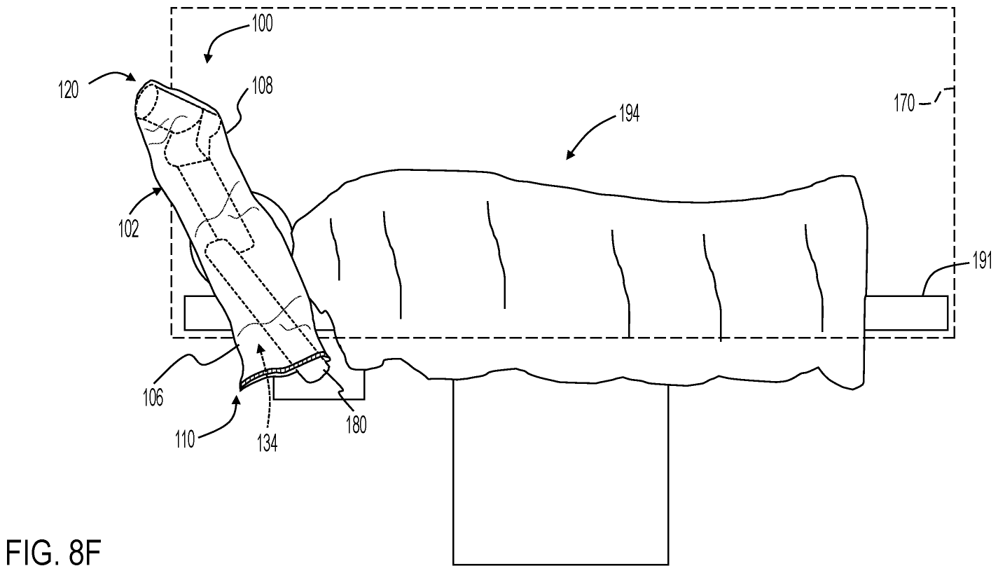

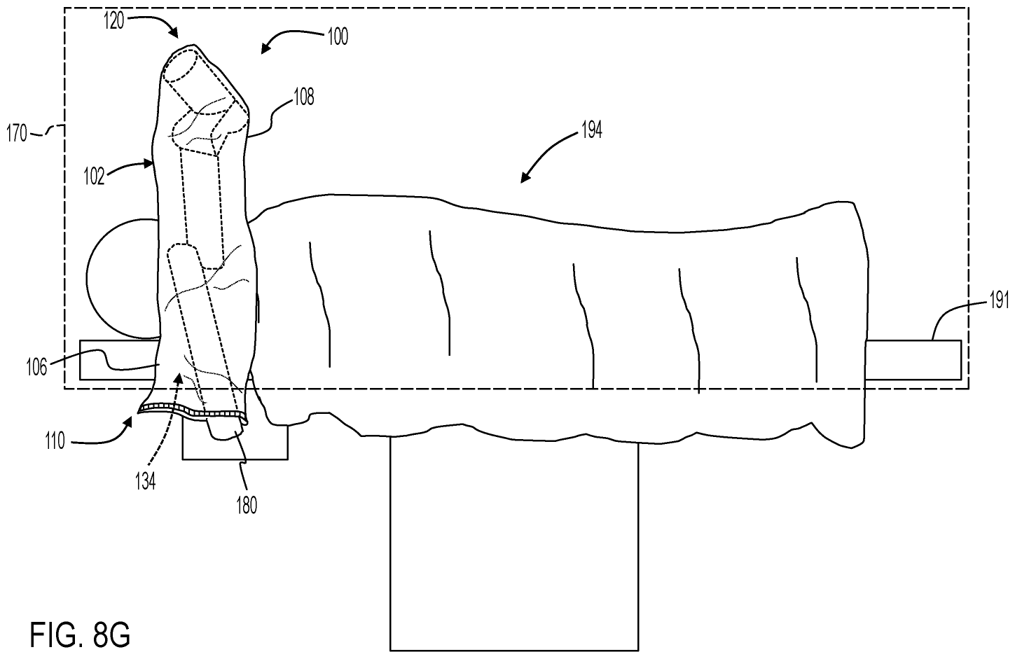

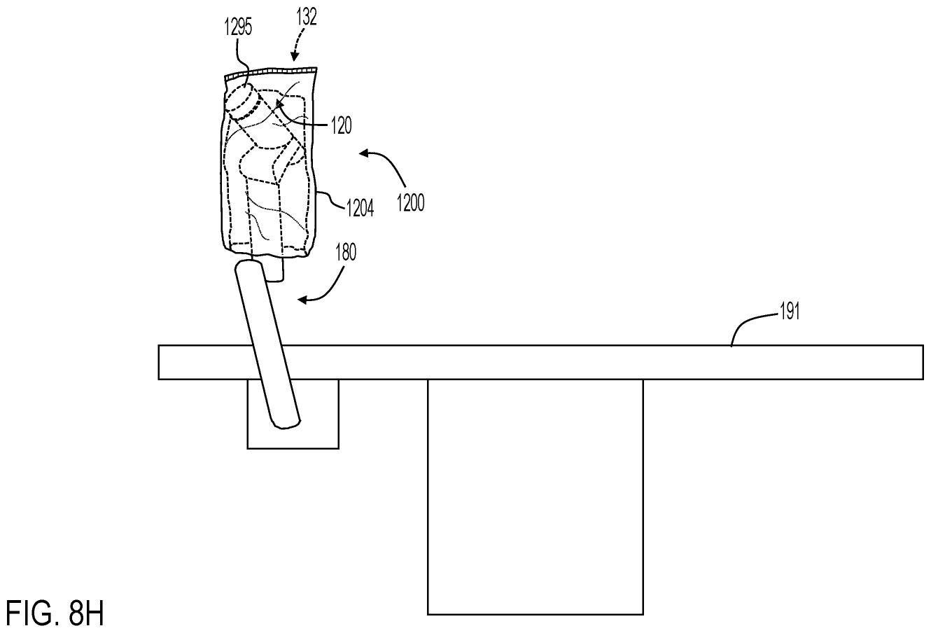

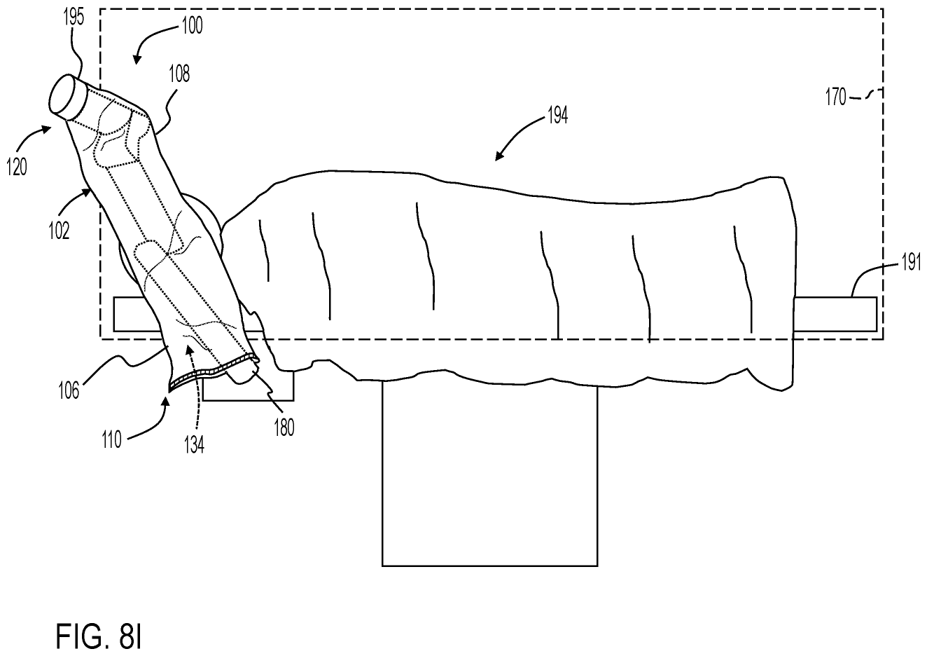

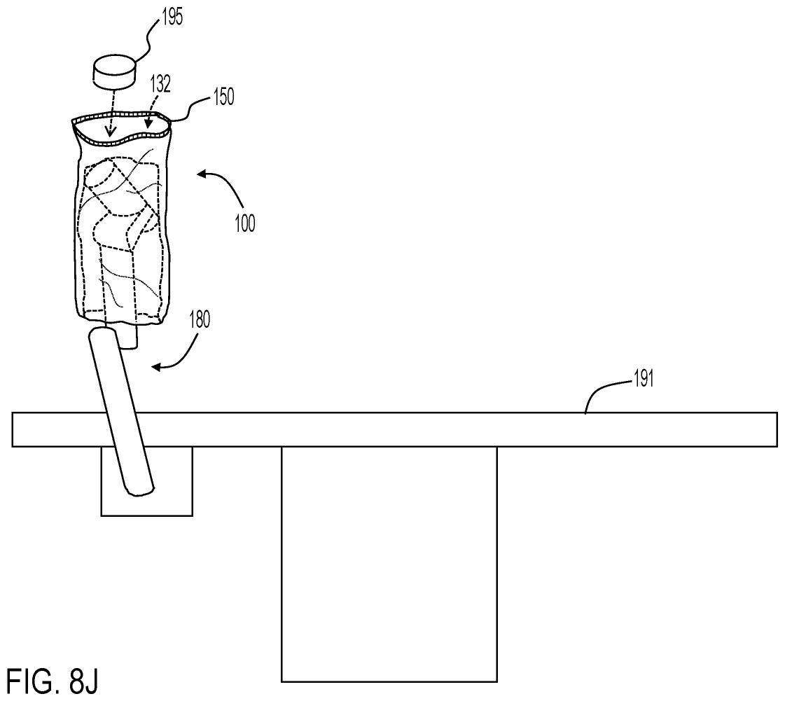

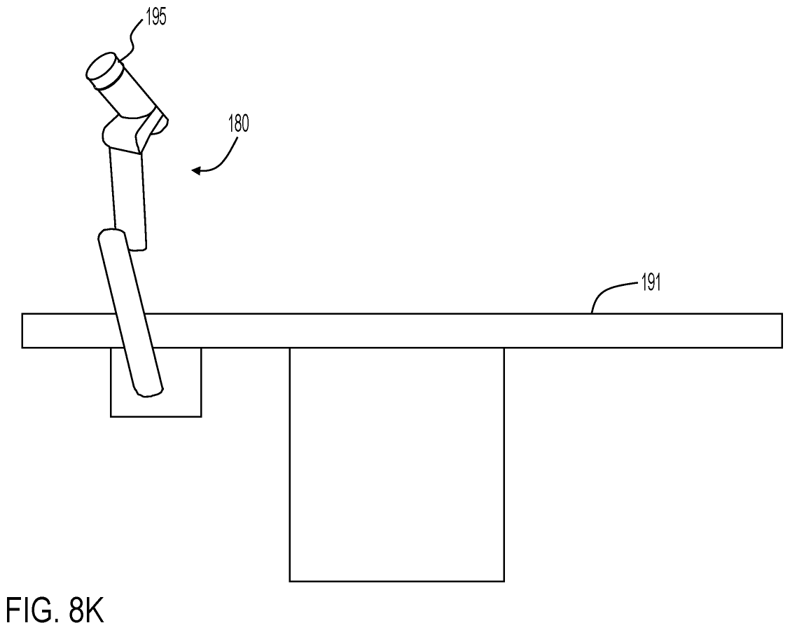

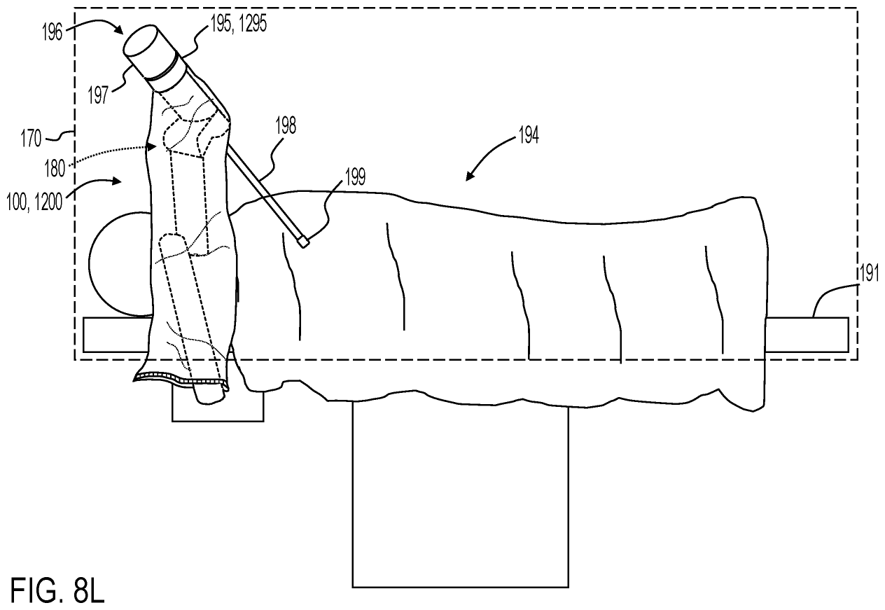

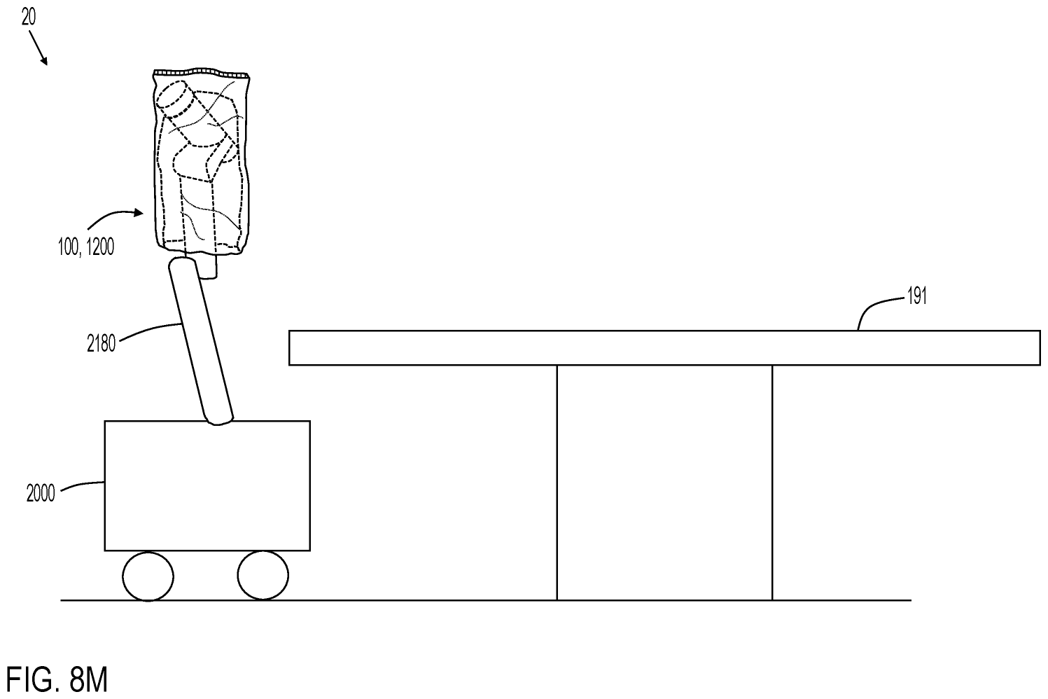

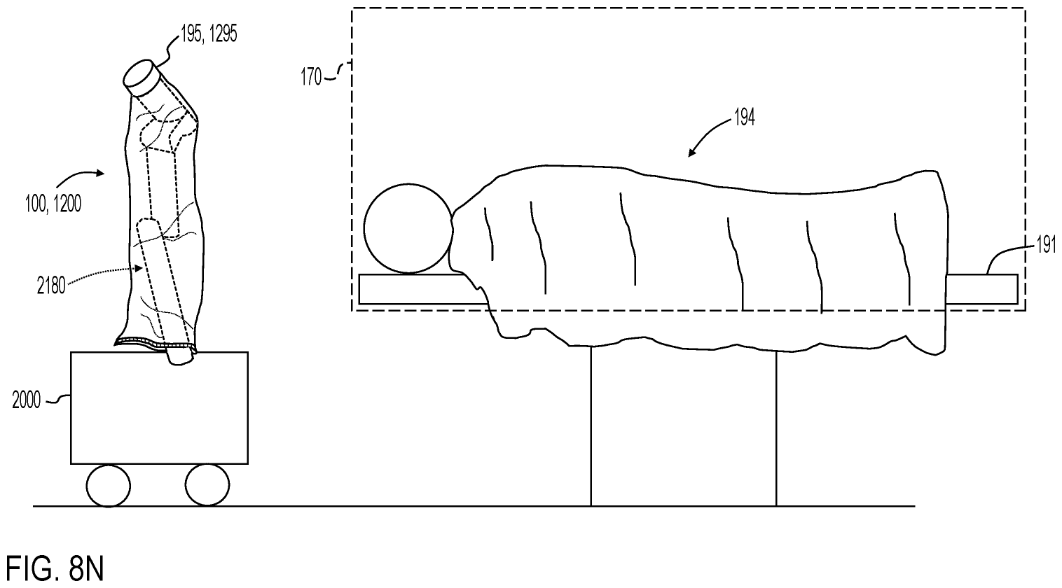

Turning now to FIGS. 8A and 8M, embodiments of systems 10 and 20 in which the sterile drape assembly 100 may be deployed will be described. Following the description of the systems 10 and 20, various methods of using the sterile drape assembly 100 in the system 10 will be described, also with reference to FIGS. 8A-8O.

In FIGS. 8A and 8M, the systems 10 and 20 are configured as a computer-assisted, teleoperable medical system, and thus are shown in connection with an operating table 191 on which a patient (not shown in FIG. 8A) is to be positioned during a medical procedure. The systems 10 and 20 in this configuration may be usable, for example, to perform any of a variety of medical procedures, such as surgical procedures, diagnostic procedures, imaging procedures, therapeutic procedures, etc. It should be understood that a teleoperable medical system is just one non-limiting embodiment of the systems 10 and 20, and the same principles discussed herein are also applicable to non-medical embodiments of the systems 10 and 20. In non-medical embodiments, the operating table 191 may be replaced with another non-medical worksite, such as a manufacturing or repair table, an assembly line, a manufacturing/production floor, etc., which may be designed to support an inanimate workpiece (something being manufactured, repaired, tested, etc.). Moreover, even when used in a medical context, the systems 10 and 20 need not be used on a living human patient. For example, a non-human animal, a cadaver, tissue-like materials used for training purposes, and so on, may be supported on the table and worked on by system 10 or 20.

As shown in FIG. 8A, the system 10 comprises a manipulator assembly 1001, a control system 1006, and a user input and feedback system 1004. The system 10 may also include an auxiliary system 1008. These components of the system 10 are described in greater detail blow. The system 10 also comprises one of the sterile drapes disclosed herein, such as the sterile drape assembly 100. The system 10 may also comprise an instrument 196, as shown in FIG. 8L. The system 10 may also comprise an instrument sterile adaptor (ISA), such as the ISA 195 or 1295, as shown in FIG. 8L.

When in use, a sterile field 170 may be designated and established around the worksite. The size, shape, and sterility requirements of the sterile field 170 may vary widely according to the different needs, rules, and best practices associated with the procedure(s) being performed and the entity performing the procedure(s). The sterile field 170 illustrated in the figures is meant merely as an example to aid in understanding the description, and is not limiting. In various figures, the sterile field 170 is illustrated to indicate a general location of where the sterile filed 170 is or where it will ultimate be located once established, but this does not necessarily mean that the sterile field 170 will have been established at the point in time illustrated in that Figure.

The manipulator assembly 1001 comprises one or more manipulators 180. FIG. 8A illustrates one manipulator 180, but any number of manipulators 180 may be included. A manipulator may comprise a single mechanical link or may comprise a kinematic structure of two or more links coupled together by one or more joints. In the embodiment illustrated in FIG. 8A, the manipulator 180 comprises a first link 181 coupled to a second link 182 by joint 184, a second link 182 coupled to a third link 183 by a joint 185, with the third link 183 coupled to an operating table 191 by a joint 186. The manipulator 180 is movable through various degrees of freedom of motion provided by the joints, thus allowing an instrument mounted thereon (see instrument 196 in FIG. 8L) to be moved around the worksite. For example, some joints may provide for rotation of links relative to one another, other joints may provide for translation of links relative to one another, and some may provide for both rotation and translation. Some or all of the joints may be powered joints, meaning a powered drive element may control movement of the joint through the supply of motive power. Such powered drive elements may comprise, for example, electric motors, pneumatic or hydraulic actuators, etc. Additional joints may be unpowered joints. The specific number and arrangement of links and joints in the manipulator 180 shown in FIG. 8A is not limiting, and in practice a manipulator may include more or fewer links and more or fewer joints, depending on the needs of the system 10. The more links and joints are included, the greater the degrees of freedom of movement of the manipulator 180.

In the embodiment of FIG. 8A, the manipulator 180 is coupled to the operating table 191. This arrangement may particularly benefit by use with the sterile drape assemblies disclosed herein, such as the sterile drape assembly 100, as this arrangement gives rise to particular challenges that the sterile drape assemblies disclosed herein may mitigate, as described above. However, the sterile drape assemblies disclosed herein can also be used in systems in which the manipulator or other equipment to be draped is not coupled to an operating table, such as for example, the type of manipulator that is part of a mobile manipulator system, which is mobile and separate from the operating table. For example, FIG. 8M illustrates schematically such a system 20 comprising a mobile base 2000, which may also be referred to as a patient-side cart, with one or more manipulators 2180 coupled to the mobile base 2000. The manipulators 2180 may be similar to the manipulators 180 described above, except for being coupled to a mobile base 2000 instead of to the operating table 191. Additional details of mobile manipulator systems that may be used as the system 20 are found, by way of nonlimiting example, in, for example, U.S. Pat. No. 9,358,074 (filed May 31, 2013) to Schena et al., entitled “Multi-Port Surgical Robotic System Architecture,” which is hereby incorporated by reference in its entirety, or any of the various manipulator systems that are part of various da Vinci® Surgical Systems, commercialized by Intuitive Surgical, Inc., of Sunnyvale, California. Moreover, although the manipulators 180 and 2180 are illustrated as one non-limiting embodiment of equipment to be draped, it should be understood that the same principles discussed herein are also applicable to other any other types of equipment that it may be desirable to drape.

Each manipulator 180 may be configured to support and operate one or more instruments 196 (see FIG. 8L). The instruments 196 may include any tool or instrument, including for example industrial instruments and medical instruments (e.g., surgical instruments, imaging instruments, diagnostic instruments, therapeutic instruments, etc.). A manipulator 180 may comprise an instrument manipulator mount 187 to which an instrument 196 can be removably coupled. An instrument manipulator mount may also be referred to in the art as an instrument holder or an instrument manipulator support. The instrument manipulator mount 187 may be located, for example, at a generally distal end of the manipulator 180. For example, the first link 181 may comprise or may be coupled to an instrument manipulator mount 187. The instrument manipulator mount 187 has an interface 188 comprising output couplers to engage (directly or indirectly via an intermediary) with input couplers of the instrument 196 to provide driving forces or other inputs to the mounted instrument 196 to control operations of the instrument 196, such as moving an end-effector of the instrument, opening/closing jaws, driving translation and/or rotation of a variety of components of the instrument, etc. The output couplers may be driven by actuators (e.g., electrical motors, hydraulic actuators, pneumatic actuators, etc.). As described in greater detail below with reference to FIGS. 8H-8L, an instrument sterile adaptor (ISA) 195 may be disposed between the instrument 196 and the instrument manipulator mount interface 188 to maintain sterile separation between the instrument 196 and the instrument manipulator mount 187. The instrument manipulator mount 187 may also comprise other interfaces (not illustrated), such as electrical interfaces to provide and/or receive electrical signals to/from the instrument 196. In some embodiments, the manipulator assembly 1001 can include flux delivery transmission capability as well, such as, for example, to supply electricity, fluid, vacuum pressure, light, electromagnetic radiation, etc. to the end effector. In other embodiments, such flux delivery transmission may be provided to an instrument through another auxiliary system 1008, described further below and as those of ordinary skill in the art would be familiar with in the context of computer-assisted, teleoperated medical systems.

The system 10 can also include a user input and feedback system 1004 operably coupled to the control system 1006. The user input and feedback system 1004 comprises one or more input devices to receive input control commands to control operations of the manipulator assembly 1001. Such input devices may include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device may be provided with the same degrees of freedom as the associated instrument that they control, and as the input device is actuated, the instrument, through drive inputs from the manipulator assembly, is controlled to follow or mimic the movement of the input device, which may provide the user a sense of directly controlling the instrument. Telepresence input devices may provide the operator with telepresence, meaning the perception that the input devices are integral with the instrument. The user input and feedback system 1004 may also include feedback devices, such as a display device (not shown) to display images (e.g., images of the workspace as captured by one of the instruments 1010), haptic feedback devices, audio feedback devices, other graphical user interface forms of feedback, etc.

The control system 1006 may control operations of the system 10. In particular, the control system 1006 may send control signals (e.g., electrical signals) to the manipulator assembly 1001 to control movement of the joints and to control operations of the instruments 196 (e.g., through a drive interface 188 at the instrument manipulator 187). In some embodiments, the control system 1006 may also control some or all operations of the user input and feedback system 1004, the auxiliary system 1008, or other parts of the system 10. The control system 1006 may include an electronic controller to control and/or assist a user in controlling operations of the manipulator assembly 1001. The electronic controller comprises processing circuitry configured with logic for performing the various operations. The logic of the processing circuitry may comprise dedicated hardware to perform various operations, software (machine readable and/or processor executable instructions) to perform various operations, or any combination thereof. In examples in which the logic comprises software, the processing circuitry may include a processor to execute the software instructions and a memory device that stores the software. The processor may comprise one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In examples in which the processing circuitry includes dedicated hardware, in addition to or in lieu of the processor, the dedicated hardware may include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry may also include any combination of dedicated hardware and processor plus software.

Although not illustrated in FIG. 8M, it should be understood that the system 20 may also comprise additional components, systems, and subsystems, such as the user input and feedback system 1004, control system 1006, and auxiliary system 1008 described above.

Differing degrees of user control versus autonomous control may be utilized in the systems 10 and 20, and embodiments disclosed herein may encompass fully user-controlled systems, fully autonomously-controlled systems, and systems having any combination of user and autonomous control. For operations that are user-controlled, the control system 1006 generates control signals in response to receiving a corresponding user input command via the user input and feedback system 1004. For operations that are autonomously controlled, the control system 1006 may execute pre-programmed logic (e.g., a software program) and may determine and send control commands based on the programming (e.g., in response to a detected state or stimulus specified in the programming). In some systems, some operations may be user controlled and others autonomously controlled. Moreover, some operations may be partially user controlled and partially autonomously controlled—for example, a user input command may initiate performance of a sequence of events, and then the control system 1006 may perform various operations associated with that sequence without needing further user input.

The auxiliary system 1008 may comprise various auxiliary devices that may be used in operation of the system 10. For example, the auxiliary system 1008 may include power supply units, auxiliary function units (e.g., functions such as irrigation, evacuation, energy supply, illumination, sensors, imaging, etc.). As one example, in a system 10 for use in a medical procedure context, the auxiliary system 1008 may comprise a display device for use by medical staff assisting a procedure, while the user operating the input devices may utilize a separate display device that is part of the user input and feedback system 1004. As another example, in a system 10 for use in a medical context, the auxiliary system 1008 may comprise flux supply units that provide surgical flux (e.g., electrical power) to instruments 1010. An auxiliary system 1008 as used herein may thus encompass a variety of components and does not need to be provided as an integral unit.

### IV. Methods of Using Sterile Drape Assembly

FIGS. 8A-8O illustrates various embodiments of methods of using sterile drape assemblies disclosed herein, such as the sterile drape assembly 100. The methods are illustrated in the context of the system 10 described above to aid understanding, but it should be understood that the same methods are also applicable to other systems and draping of other pieces of equipment.

A first method of using the sterile drape assemblies disclosed herein is illustrated in FIGS. 8A-8G. In particular, the method will be described with reference to the drape assembly 100 described above with respect to FIGS. 1 and 2. FIG. 8A illustrates a first state, in which the piece of equipment to be draped, the manipulator 180, is ready for draping but is not yet draped. FIG. 8B illustrates a second state in which the manipulator 180 (or other equipment) has been partially draped as part of an initial draping procedure. The initial draping comprises providing the sterile drape assembly 100 in the partially inverted state and covering part of the manipulator 180 with the partially inverted drape assembly 100 by inserting the distal (free) end of the manipulator 180 through the opening 135 into the partially enclosed interior volume 134 defined by the partially inverted drape assembly 100 (see also FIGS. 1B and 1C). The initial draping step may be performed before preparations of the worksite have commenced, such as before a patient 194 is prepared on the operating table 191. As described above, this may greatly ease the process of installing the sterile drape assembly 100.

As shown in FIG. 8C, once the partially inverted drape assembly 100 has been partially installed on the manipulator 180 and any other operations associated therewith are completed, the manipulator 180 and drape assembly 100 may be stowed away outside of the region that will ultimately become the sterile field 170, as described above. This may enable preparation of the worksite, including for example the positioning and preparation of a patient 194 on the operating table 191, as shown in FIG. 8C. The preparation of the worksite may also include the establishing of the sterile field 170 by sterilizing or draping any exposed surfaces in the intended region for the field 170 that are not already sterile. The positioning of the drape assembly 100 outside of the region that is or will ultimately become the sterile field 170 compromises the sterility of the exterior surfaces of the drape assembly 100, specifically the portion of the surface 101 that is exposed to the exterior environment. In FIGS. 8A-8J, compromised surface portions of the drape assembly 100 are indicated with dotted shading, whereas sterile surfaces of the drape assembly 100 are shown without dotted shading.

As shown in FIG. 8D, after preparations of the worksite are complete, the manipulator 180 is moved to a position/pose at which the drape assembly 100 can be converted to the fully inverted state. This position/pose of the manipulator 180 may be, for example, one in which the manipulator 180 is fully or partially extended to facilitate draping but which is outside of the sterile field 170. For example, the manipulator 180 may be extended to lean outwardly away from an operating table 191, thus avoiding the sterile field 170 that may be located in the region above and near the operating table 191. A process of converting the drape assembly 100 from the partially inverted state to the fully inverted state may then be performed. As shown in FIG. 8E, this process of converting the drape assembly 100 to the fully inverted state comprises releasing the closure mechanism 150 to open the first end 110 of the drape assembly 100. With the first end 110 opened, the first end 110 is then moved along the length of the manipulator 180, as shown in FIG. 8E, to progressively cover the remainder of the manipulator 180 and to progressively uncover the portion 108 of the drape assembly 100 that was previously covered by the portion 106. This results in progressively exposing more of the sterile surface 102 of the drape assembly 100 to the exterior environment, while progressively inverting the portion of surface 101 that was exposed to the environment outside the sterile field 170 such that it faces into the partially enclosed interior volume 134 in which the manipulator is received instead of facing externally into the sterile field 170. As shown in FIG. 8F, the conversion of the drape assembly 100 to the fully inverted state is completed once the first end 110 has been fully extended such that inner portion 108 is no longer covered by the outer portion 106 and the entirety of the sterile surface 102 is exposed to the exterior environment while the entirety of the surface 101 faces inward and toward the manipulator 180. Thus, in the fully inverted state, the sterile drape assembly 100 will no longer compromise the sterility of the sterile field 170 because only sterile surface portions thereof are exposed. Thus, after conversion of the drape assembly 100 into the fully inverted state, the manipulator 180 may be moved into the sterile field 170, as shown in FIG. 8G.

In other embodiments (not illustrated), instead of converting the drape assembly 100 to the fully inverted state at a position that is outside the ultimate boundaries of the sterile field 170 and then moving the draped manipulator 180 into the sterile field 170 as described above, the manipulator 180 is moved to a position that will ultimately be inside of the sterile field 170 and is converted there to the fully inverted state. In some of these embodiments, the sterile field 170 is not yet established while the drape conversion process is ongoing and is established only after completion of the drape conversion. In these embodiments, once the sterile field 170 is established, it encompasses the now fully-draped manipulator 180 without requiring repositioning of the manipulator 180. In other embodiments, the sterile field 170 is already established prior to the conversion of the drape assembly 100 but the sterile field 170 initially encompasses a smaller region than what its ultimate boundaries will encompass such that the undraped manipulator 180 is outside of the sterile field 170 while the drape assembly 100 is being converted to the fully inverted state. The sterile field 170 is then expanded after completion of the conversion process to encompass the now fully-draped manipulator 180.

In still other embodiments, the partially draped manipulator 180 may be brought into the sterile field 170 and converted into the fully-inverted state while in the sterile field 170. While the presence of the compromised exposed surfaces of the drape assembly 100 and manipulator might compromise the sterility of the sterile field 170 under some regulations or best practices, in some circumstances and under some regulations or best practices the transient presence of the compromised exposed surfaces in the sterile field 170 may be allowed without compromising the sterility of the field 170 under certain conditions, such as that the drape be converted to the fully inverted state without coming into contact with other sterile surfaces in the field.

In some embodiments, an instrument sterile adaptor (ISA) is also provided to facilitate mounting of an instrument to the manipulator 180. The ISA is configured to receive an instrument 196 mounted thereon, as shown in FIG. 8M. The ISA may comprise intermediate couplers that interface between the output couplers of the interface 188 of the instrument manipulator mount 187 and corresponding input couplers of the instrument 196 to transfer driving forces therebetween. The output couplers, intermediate couplers, and input couplers may all be referred herein generically as couplers when distinguishing between them is not necessary. The ISA may also have attachment features that attach to the instrument 196 to hold the instrument 196 on the instrument manipulator mount 187. The ISA is configured to provide a sterile barrier at the interface between the instrument 196 and the interface 188 of the instrument manipulator mount 187. The ISA may be beneficial in some circumstances because it may be configured to provide a sterile barrier that is robust enough to handle the stresses that may be associated with the motion of the couplers, whereas some sterile drapes might not be configured to handle such stresses (e.g., the moving parts might cause some sterile drapes to tear). In addition, other sterile adaptors (not illustrated) may also be provided, such as an adaptor/interface to grasp a cannula through the drape. Although FIGS. 1-7, 9-14B, and 17A-18D do not show an ISA or other sterile adaptor as a part of (or as being coupled to) the drape assemblies illustrated therein, it should be understood that in various embodiments an ISA (such as the ISA 195 or 1295) or other sterile adaptor could be part of, or could be coupled to, these drape assemblies. In such embodiments the ISA or other sterile adaptor would be coupled to the drape body 104 at the second end 120 of the drape assembly 120, and would protrude into the interior volume 132 when the drape assembly 100 is in the partially inverted state.

In some embodiments, the ISA or other sterile adaptor is part of the drape assembly 100, while in other embodiments the ISA or other sterile adaptor is coupled with the drape assembly 100 before or during installation of the drape assembly 100. Various examples of these embodiments are described below with reference to FIGS. 8H-8L.

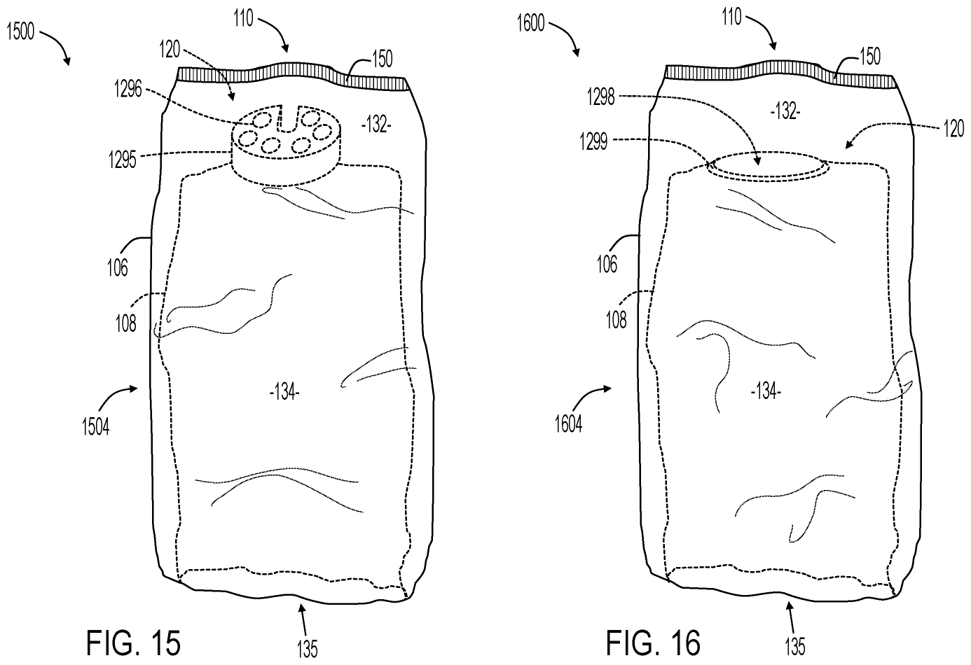

In embodiments in which the ISA or other sterile adaptor is part of the sterile drape assembly or is coupled with the sterile drape assembly prior to installation, the process of installing the sterile drape assembly on the manipulator 180 may be similar to that described above, except that the ISA or other sterile adaptor may be mounted to the manipulator interface 188 during one of the steps of the process, as shown in FIG. 8H. Such drape assemblies that have an ISA or other sterile adaptor coupled thereto (whether at manufacture or in the field prior to installation of the drape assembly) may be referred to herein as a drape assembly 1200. An example of such a drape assembly is described herein in which the sterile adaptor is an ISA, which may be referred to herein as an ISA 1295. An embodiment of the drape assembly 1200 and ISA 1295 is described in greater detail below with reference to the embodiment of FIG. 15. Because the ISA 1295 is coupled to the drape body 1204 in these embodiments, the ISA 1295 is already present in the interior volume 132 when the drape body 1204 is draped over the manipulator 180 in the partially inverted state. Thus, after the manipulator 180 is received within the drape body 1204 of the drape assembly 1200 during the initial installation process, the ISA 1295 can be positioned on and coupled to the manipulator interface 188. For example, FIG. 8H illustrates an embodiment in which a drape assembly 1200 comprising an ISA 1295 is installed on the manipulator 180, with the ISA 1295 being mounted to the manipulator interface 188 prior to stowing the manipulator 180, i.e., between the states illustrated in FIGS. 8B and 8C. The remaining operations for installing the drape assembly can be similar in this embodiment to those already described above. Mounting the ISA 1295 to the manipulator 180 prior to preparing the worksite, as occurs in these embodiments, can be beneficial in some circumstances because installing an ISA can be one of the more difficult and/or time-consuming parts of the overall installation process, and thus doing this step when there is less chance for interference with the worksite can ease the process. When the manipulator 180 is ultimately stowed after mounting of the ISA 1295 and partial installation of the drape assembly 1200, the ISA 1295 will be protected from contamination because it is covered drape body 1204.

Turning now to FIGS. 8I-8L, various other embodiments in which the ISA is coupled to the drape assembly 100 during installation of the drape assembly (rather than prior to installation of the drape assembly 100) will be described below. As shown in FIG. 8I, in some embodiments, an ISA is mounted to the manipulator interface 188 over the sterile drape assembly 100 after the sterile drape assembly 100 has been fully inverted (e.g., after the state illustrated in FIG. 8F or after the state illustrated in FIG. 8G). As shown in FIG. 8J, in other embodiments the ISA 195 is mounted to the manipulator interface 188 while the sterile drape assembly 100 is partially installed and prior to stowing of the manipulator 180 outside of the sterile field 170 (between the states shown in FIGS. 8B and 8C), for example by opening the closure mechanism 150 (if not already open) to introduce the ISA 195 into the interior volume 132, mounting the sterile adaptor 195 over the drape body 104, and then closing the closure mechanism 150 after the ISA 195 has been mounted to the manipulator interface 188. Subsequent operations may be the same as those described above. In some of the embodiments described above in which the ISA 195 is mounted on the drape body 104, the ISA 195 is positioned over one or more corresponding openings in the drape body 104 so that drape material is not disposed between couplers of the ISA 195 and the manipulator interface 188, while in other embodiment there are no such openings and the couplers engage one another with the drape material disposed therebetween. As shown in FIG. 8K, in other embodiments the ISA 195 is mounted on the manipulator 180 prior to the drape assembly 100 being installed, in which case the ISA 195 may positioned under the drape assembly 100 once it is installed. In some such embodiments, the couplers of the ISA 195 may engage with couplers of the instrument through the drape body 104 (e.g., through openings in the drape body 104 or with the drape body 104 disposed between the couplers).

Regardless of how or when the ISA 195 or 1295 is mounted to the manipulator 180, once the drape assembly 100 or 1200 has been converted to the fully inverted state and the ISA 195 or 1295 has been mounted, the instrument 196 may then be mounted to the ISA 195 or 1295 as shown in FIG. 8L. The instrument 196 comprises a shaft 198, an end effector 199 supported by a distal end portion of the shaft 198 and configured to preform one or more functions (e.g., cutting, sealing, grasping, stapling, image capture, irrigation, etc.), and a force transmission assembly 197 configured to mount to the ISA 195, 1295 or to the manipulator interface 188 and to transfer drive forces received therefrom to control degrees of freedom of motion and/or functions of the instrument 196. In some embodiments, the instrument 196 is mounted prior to bringing the manipulator into the sterile field 170 (or prior to establishing or expanding the sterile field 170 to encompass the manipulator 180), while in other embodiments the instrument 196 is mounted while the manipulator 1870 is in the sterile field 180.

In some embodiments, the ISA 195 may be omitted. For example, in some embodiments, the sterile drape assembly 100 may be configured to provide a sufficiently robust sterile barrier at the interface between the instrument 196 and the manipulator 180. In such embodiments, the instrument 196 is coupled to the manipulator 180 with only the drape assembly 100 therebetween.

In some embodiments, the drape assembly 100, 1200 may comprise auxiliary features (not illustrated) that provide additional functionalities, such as ties, tape strips, Velcro straps, and/or cable guides, to organize and/or hold cords in place (e.g., the cables for energy instruments or endoscope light fibers and data connections), or tie straps or elastomeric bands to bundle excess drape material closer to the enclosed equipment.

After installing the ISA 195 or 1295 according to any of the methods described above, various additional associated operations may also be performed. For example, in some embodiments an initialization process may be performed once physical coupling of the ISA 195 to the manipulator 180 has occurred. The initialization process may comprise, in some embodiments, arranging the intermediate couplers of the ISA 195 relative to corresponding output couplers of the interface 188 of the instrument manipulator mount 187 such that complementary engagement features of the intermediate couplers and output couplers appropriately engage. In some embodiments, the initialization process may be performed by the control system 1006 (see FIG. 8A) in response to the ISA 195 or 1295 being installed. For example, the control system 1006 may drive the output couplers to move in a predetermined manner that causes the engagement of the complementary engagement features.

Turning now to FIGS. 8M-8O, the installation of the drape assembly 100, 1200 in the system 20 will be described. The operations for installing the drape assembly 100, 1200 in the system 20 are similar to those described above in relation to the system 10, except that in the system 20 the mobile base 2000 can be moved away from the operating table 191 while the worksite is being prepared instead of, or in addition to, stowing the manipulator 2180. Thus, as shown in FIG. 8M, the drape assembly 100, 1200 in the partially inverted state may be inserted on the manipulator 2180 while the manipulator is positioned away from the operating table 191. In some embodiments, an ISA 195 or 1295 may also be mounted to the manipulator 2180 at this point, in any of the ways already described above in relation to the system 10. Thereafter, once the preparations of the worksite have been completed, the sterile drape assembly 100, 1200 may be converted to the fully inverted state, as shown in FIG. 8N, in the same manner as described above in relation to the system 10. Once the drape assembly 100, 1200 has been fully inverted, the manipulator 2180 may be brought into the sterile field 170, as illustrated in FIG. 8O. The instrument 196 may be mounted on the ISA 195 or 1295 either prior to moving the manipulator 2180 into the sterile field 170 in some embodiments or after moving the manipulator 2180 into the sterile field 170 in other embodiments.

In some embodiments, a drape body of a drape assembly is positioned between the ISA and the instrument being mounted to the manipulator, as described above. In some of these embodiments, a portion of the drape body that is to be positioned between the ISA and the instrument or between the ISA and the manipulator and the instrument may be configured to be more resilient (e.g., thicker, stronger, reinforced, etc.) so as to withstand motion of the couplers between which the drape body is sandwiched. In other embodiments, one or more holes are provided in the second end of the drape body at locations corresponding to the ISA couplers so as to allow for the couplers of the ISA, instrument, and/or manipulator to engage one another without portions of the drape body being sandwiched therebetween. One embodiment of a drape assembly with such a hole in a drape body is described in greater detail below with reference to FIG. 16.

### V. Additional Drape Assembly Configurations/Mechanisms

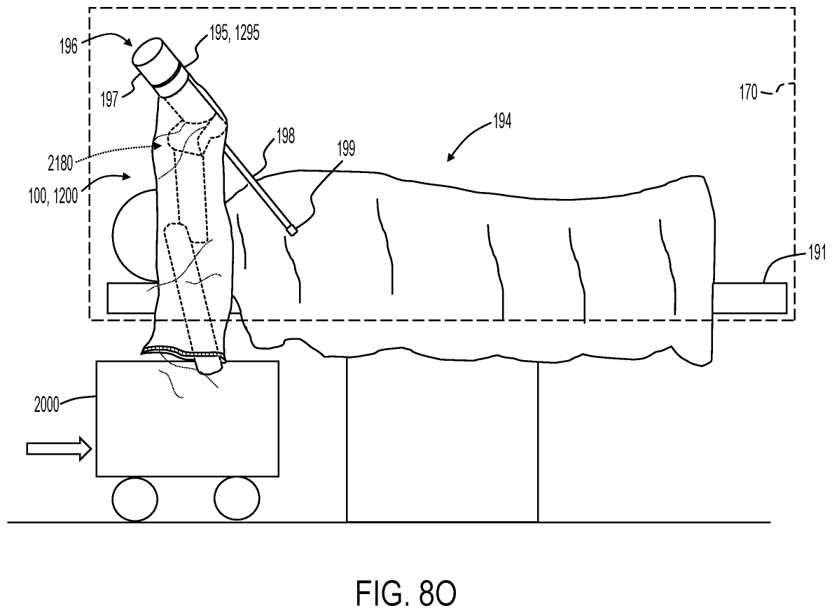

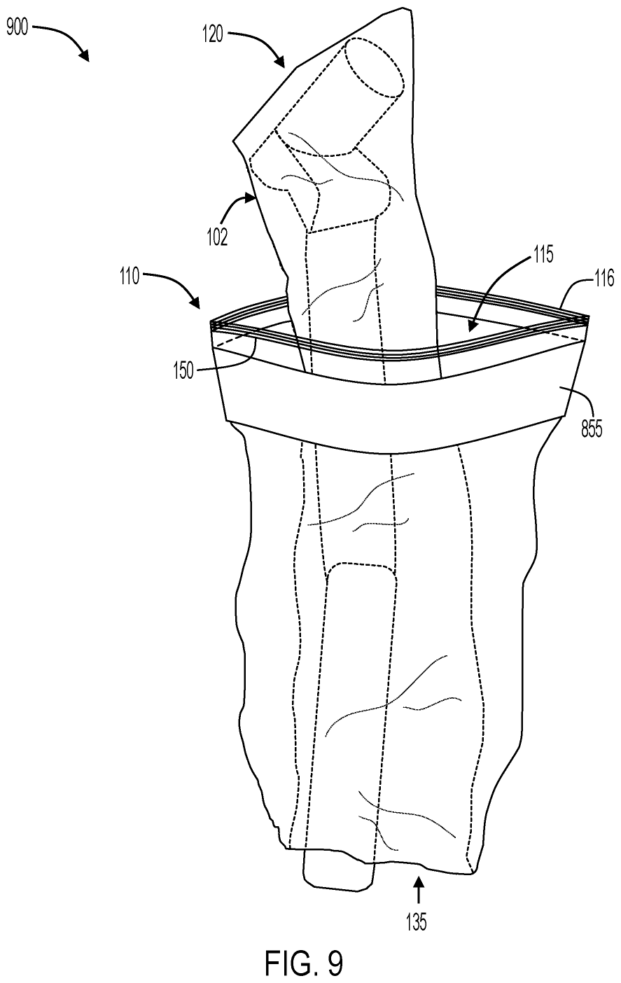

Various additional configurations and/or components may be added to sterile drape assemblies as described above, or existing features of a sterile drape assembly modified, to add additional functions or improve existing functions of the sterile drape assembly. Some of these additional features and/or modifications are illustrated in FIGS. 9-16, which illustrate various embodiments of sterile drape assemblies that can be used as the sterile drape assembly 100 described above. The same reference numbers are used herein and in the Figures to designate similar or the same components amongst the sterile drape assembly 100 and the embodiments of FIGS. 9-16. The descriptions of the components of the sterile drape assembly 100 above are applicable to the similar or same components of the embodiments of FIGS. 9-16, and therefore duplicative description thereof is omitted. Although each of the embodiments illustrated in FIGS. 9-16 highlights particular configurations and/or components in isolation, it should be understood that multiple of these additional components and/or configurations could be combined together. In particular, embodiments are contemplated herein comprising each and every possible combination of the various embodiments illustrated in FIGS. 9-16 and described below, except where noted otherwise, logically contradictory, or physically impossible, as would be evident to one having ordinary skill in the art.

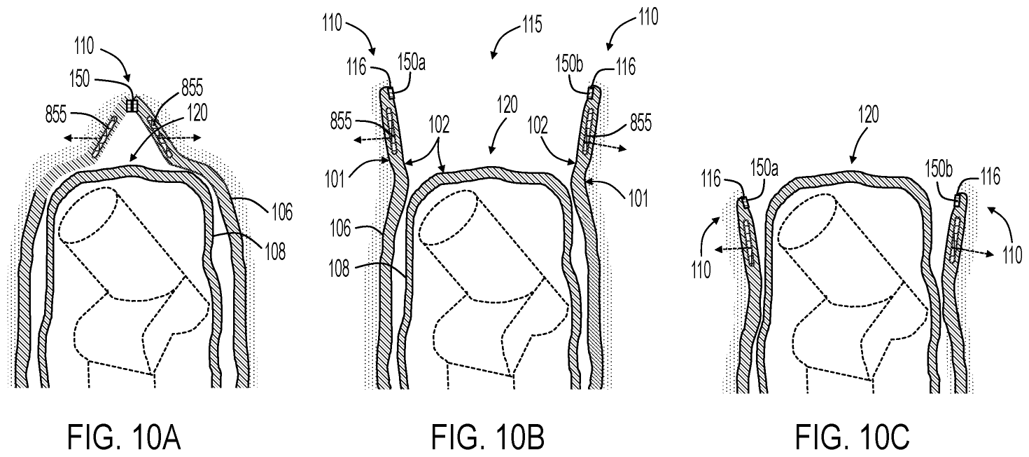

FIGS. 9-10C illustrate an embodiment of a sterile drape assembly 900 comprising a hold-open device 855. The hold-open device 855 comprises a flexible material coupled to the first end 110 of the drape 900. The hold-open device 855 is configured to hold the first end of the drape 900 widely open as the drape is being converted from the partially inverted state to the fully inverted state, i.e., as the first end 110 is moved along the length of the draped equipment to complete the draping of the equipment. This is done in order to prevent or reduce contact between the sterile surface 102 and the portion of the first end 110 forming the rim 116 of the opening 115. For example, FIGS. 10B and 10C show the first end 110 at two positions as it is moved down the draped equipment, with the hold-open device 855 holding the rim 116 away from the sterile inner surface 102 as the first end 110 is moved past.

The reason it may be desirable under some circumstances to keep the rim 116 away from the sterile inner surface 102 is to prevent contamination of the sterile inner surface 102 by the rim 116. As described above, when the drape 900 is stowed outside of the sterile field, the sterility of the surface 101 becomes compromised. Such a compromised portion is indicated in FIGS. 10A-10C by dotted shading near potentially contaminated surfaces due to exposure outside of the sterile field. As shown in FIG. 10A, all of the exposed portions of the surface 101 become compromised, and as shown in FIG. 10B this can include the portion of the first end 110 that will ultimately form the rim 116 of the opening 115 once the closure mechanism 150 is opened. In FIGS. 9-10C, the rim 116 is adjacent to, and indeed may comprise portions of, the closure mechanism 150. In particular, the closure mechanism 150 illustrated in FIGS. 9-10C separates into two parts 150a and 150b once the closure mechanism 150 is opened, and a portion of each of these parts 150a and 150b forms a part of the rim 116. In other embodiments, the rim 116 is not necessarily adjacent to or part of the closure mechanism 150. Because the rim 116 is compromised (i.e., potentially contaminated), if the rim 116 comes into contact with the sterile surface 102 while the first end 110 is being moved over the surface 102, the sterility of the surface 102 might also be compromised. Thus, to prevent or reduce such contact and the resultant potential compromise of the surface 102, the hold-open device 855 is designed to hold the rim 116 of the opening 115 away from the inner surface 102 (to the extent possible). In other words, the hold-open device 855 holds the opening 115 such that its perimeter is around and sufficiently spaced from the portion 108 of the drape body 104 so as to minimize contact between the rim 116 of the opening 115 and the surface 102 as the first end 110 is moved along the length of the portion 108 and over the remaining portion of the equipment to be draped.

In some embodiments, the hold-open device 855 is spring like, meaning it is substantially elastically deformable within its intended range of motion. This means that the hold-open device 855 will tend to return to its default or resting shape after being deformed. Specially, when the first end 110 is closed via the closure mechanism 150, this may partially deform the hold-open device 855, as shown in FIG. 10A. This results in spring forces pressing outward, as indicated by the dashed arrows in FIG. 10A, which urge the hold-open device 855 and the opening 115 toward the open state. As shown in FIG. 10B, when the closure mechanism 150 is released, the spring forces generated by the hold-open spring device 855 return it to its default or resting shape, thereby forcing open the opening 115.

The default or resting shape of the hold-open spring device 855 is a shape that corresponds to the opening 115 having a perimeter sufficient to provide a radial spacing between the edge of the drape body 104 forming the opening and the portion 108 of the drape body 104. In other words, the default or resting shape of the hold-open spring device 855 is a shape that results in the rim of the opening being held away from the inner surface 102. For example, the default or resting shape of the hold-open spring device 855 may be roughly annular-shaped (though not necessarily precisely circular) in profile when viewed from a perspective on a longitudinal axis of the drape body 104. For example, the default or resting shape of the hold-open spring device 855 may be elliptical, or may be have one or more relatively angular portions coupled by curved portions (like a tear drop). The hold-open spring device 855 may also have an irregular shape. The precise shape of the hold-open spring device 855 is not limited as long as the hold-open spring device 855 is able to assist in holding at least part of the rim 116 of the opening 115 away from the inner surface 102. In some embodiments, the default or resting shape of the hold-open spring device 855 also may result in a slight angling of the hold-open spring device 855, and the portion of the drape body 140 associated therewith, away from the inner surface 102, as shown in FIG. 10C. This can further reduce the likelihood of contact between the rim 116 and the inner surface 102.

In some embodiments, the hold-open device 855 is configured to have a stable closed configuration, meaning that the hold-open device 855 will generally not leave the closed configuration without the application of an external force to urge it to leave the closed configuration. However, if the hold-open device 855 is moved sufficiently towards an open configuration, the hold-open device 855 may then spring or snap the rest of the way into the open configuration and the hold-open device 855 may thereafter resist being removed from the open configuration. The springing or snaping into the open configuration and the subsequent resistance to moving out of the open configuration may be the result of internal forces (e.g., spring forces) generated by hold-open device 855.

As noted above, in some embodiments the hold-open spring device 855 is configured to exhibit spring-like properties. In some embodiments, it may be desirable for the spring force generated by the hold-open spring device 855 to be sufficiently strong to urge the opening 115 to open and to hold it in the open state, but sufficiently weak as to not tear the drape, inadvertently open the closure mechanism 150, or to make closing the closure mechanism 150 too difficult. In some embodiments, the hold-open spring device 855 may be formed by one or more strips of flexible plastic, spring steel, shape memory alloy, or the like.

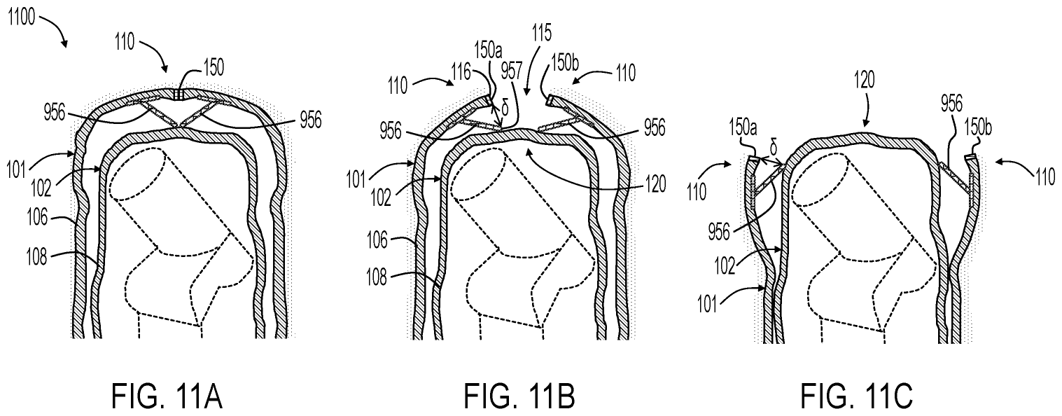

FIGS. 11A-11C illustrate an embodiment of a sterile drape assembly 1100 comprising a wiper device 956. The wiper device 956 comprises a material coupled to the first end 110 of the drape 900 and positioned on the surface 102 of the portion 106 of the drape body, as shown in FIG. 11A. Much like the hold-open spring device 855, the wiper device 956 is configured to prevent or reduce contact between the rim 116 and the sterile surface 102 as the first end 110 is moved down portion 108 and the surface 102. However, instead of doing this by holding the opening 115 open by spring force, the wiper device 956 is configured to reduce contact between the rim 116 and the surface 102 by enforcing a physical separation of the two. Specifically, the wiper device 956 is attached to the first end 110 near the rim 116 and configured to hold the rim 116 at a distance δ from a free end 957 of the wiper 956, as shown in FIG. 11B. The free end 957 of the wiper 956 comes into contact with the surface 102 occasionally or continuously as the first end 110 is moved past the portion 108 and surface 102, thus preventing the inner surface 102 from coming any closer to the rim than the distance δ.

In some embodiments, the wiper 956 comprises a single member that extends around all or part of the perimeter of the opening 115. In other embodiments, the wiper 956 comprises a multiple distinct member that are spaced apart from one another and collectively extend around all or part of the perimeter of the opening 115.

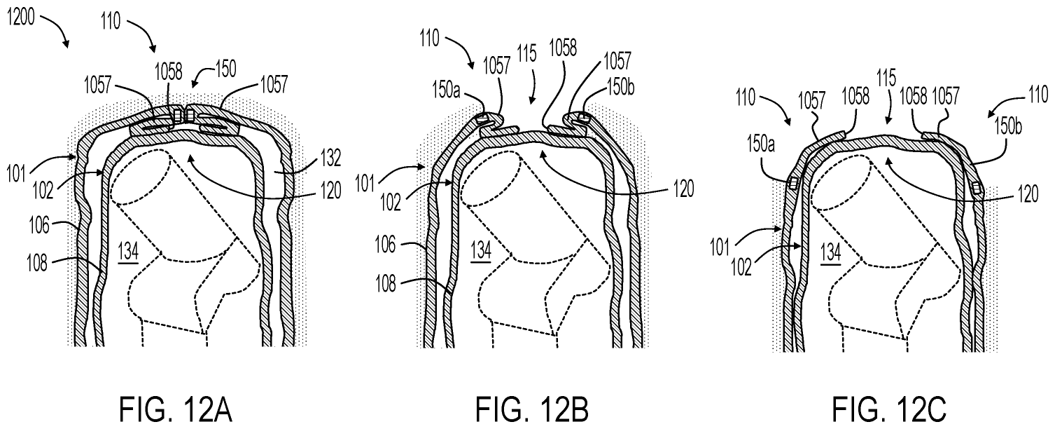

FIGS. 12A-12E illustrate an embodiment of a sterile drape assembly 1200 comprising a pleated portion 1057. The pleated portion 1057 addresses the same problem as the hold-open spring device 855 and the wiper device 956, but in a different way. Rather than holding the rim 116 away from the surface 102, the pleated portion 1057 is instead configured to protect the rim 116 of the opening from contamination during the stowing of the drape assembly 1200 outside the sterile field 170, thus obviating the need to hold the rim 116 away from the surface 102 because contact with the rim 116 is no longer going to potentially contaminate the surface 102. Specifically, as shown in FIG. 12A, the pleated portion 1057 comprise a portion of the drape material at the first end 110 that extends beyond the closure mechanism 150 and that, in the partially inverted state, is tucked into the enclosed interior volume 132 of the drape 1200 between the portion 106 and the portion 108. Because the pleated portion 1057 is contained within the interior volume 132, it is protected from being exposed to a non-sterile environment when the drape assembly 1200 is stowed or moved to such environment (as in embodiments described above, in FIGS. 12A-12C, compromised surfaces are indicated by dotted shading adjacent to the surface). When the closure mechanism 150 is opened, as shown in FIG. 12B, the rim of the opening 115 may initially comprise (or may be adjacent to) the two separated parts 150a and 150b of the closure mechanism, but as the first end 110 begins to move down past the portion 108 of the drape body, the pleated portion 1057 begins to unfold and becomes the rim of the opening 115. In particular, as shown in FIG. 12C, the edge 1058 of the pleated portion 1057 eventually becomes the rim of the opening 115. Because the edge 1058 was protected from exposure to a non-sterile environment in the stowed state it remains sterile and uncompromised. Therefore, contact of the edge 1058 with the inner surface 102 does not contaminate the inner surface 102.

In FIGS. 9-11C the closure mechanism 150 is shown as a press-fit type closure, while in FIGS. 12A-12C, the closure mechanism 150 is shown as a magnetic type closure, but the embodiments disclosed in FIGS. 9-12C are compatible with any of the closure mechanisms 150 disclosed herein, including any of those described above with reference to FIGS. 3-7.

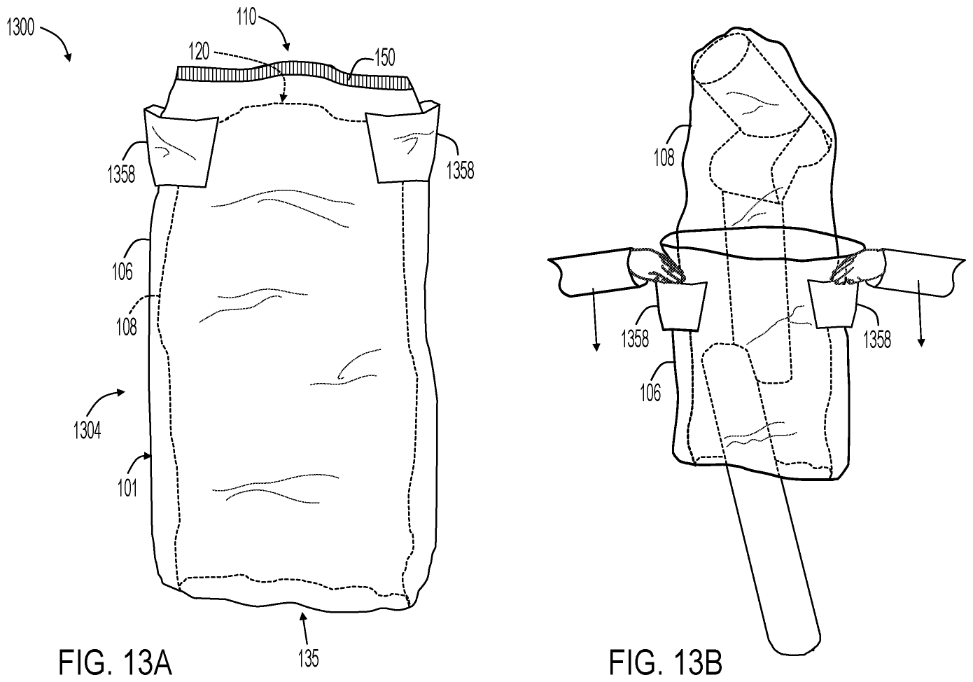

FIGS. 13A-13B illustrate another embodiment of a sterile drape assembly 1300. The sterile drape assembly 1300 comprises one or more handles 1358. The handles 1358 are arranged on the first surface 101 of the portion 106 near the first end 110. The handles 1358 allow a user to more easily grasp the first end 110 of the drape body 1304 and pull it down from the partially inverted position to complete the inversion of the drape 1300. The handles 1358 may also make it easier for a user with non-sterile hands to complete the inversion of the drape assembly 1300 without compromising its sterility by giving the user something the grasp other than the rim of the opening 115, since grasping the rim of the opening 115 might risk contaminating the surface 102. The handles 1358 may be formed, for example, as pockets attached to or formed integrally with the drape material, but such configuration is non limiting and other types of handle could be provided. In some embodiments, two handles 1358 are provided on opposite sides of the drape body 1304 to allow for even application of the downward force to the first end 110. In some embodiments, additional handles 1358 may be provided. For example, a second set of handles 1358 (not illustrated) may be provided on the surface 101 at a bottom portion (e.g., bottom half) of the portion 106. This second set of handles 1358 may facilitate the initial installation of the drape assembly 1300 on equipment by giving a user something near the opening 135 to grasp when pulling the partially inverted drape body 1304 onto the equipment.

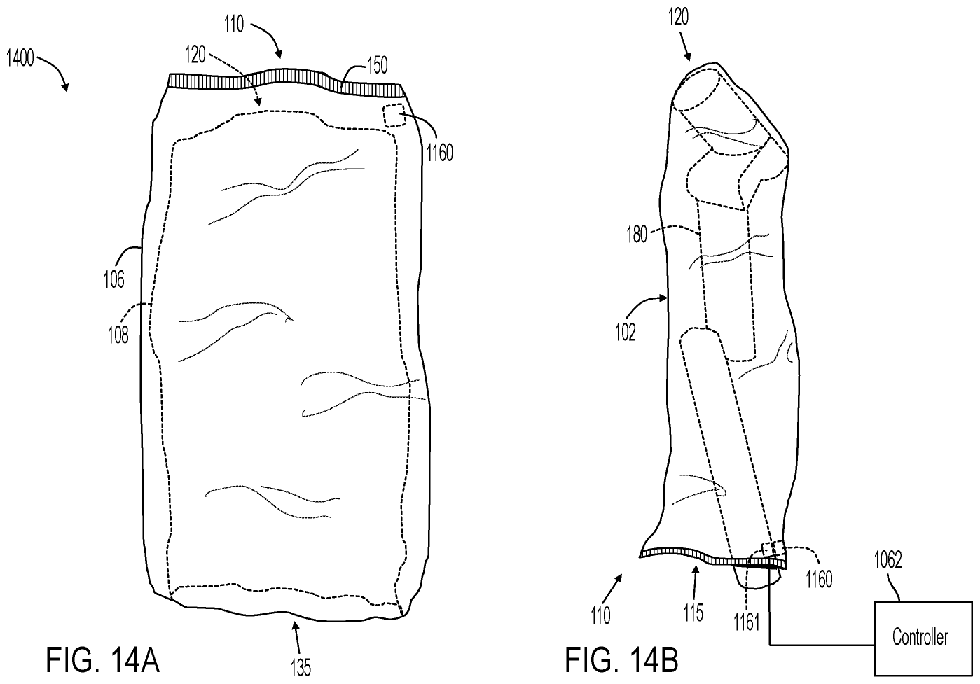

FIGS. 14A and 14B illustrate yet another embodiment of a sterile drape assembly 1400. The sterile drape assembly 1400 comprises a drape installation indicator 1160. The drape installation indicator 1160 is coupled to the portion 106 of the drape assembly 1400 near the first end 110 of the drape body 104. The drape installation indicator 1160 is positioned such that it will be located within sensing proximity to a sensing device 1161 on the draped equipment when the drape assembly 1400 is installed on the equipment and in the fully extended (fully inverted) position. When the sensing device 1161 senses the presence of the drape installation indicator 1160, it may send a signal indicating this to a controller 1062. The controller 1062 may determine that the drape 1300 is installed and in the fully inverted state (i.e., fully installed on the equipment) in response to receiving the signal from the sensing device 1161 indicating the sensed presence of the drape installation indicator 1160. The controller 1062 may then perform one or more actions based on determining that the drape assembly 1300 is installed and in the fully inverted state. For example, the controller 1062 may prevent certain operations, such as insertion of an instrument or deployment of a function of an instrument, from being performed until it determines the drape assembly 1300 is installed and in the fully inverted state, and upon determining that the drape assembly 1300 is installed the controller 1062 may allow those operations to proceed. As another example, the controller 1062 may generate an audible or visible indicator that the drape assembly 1300 is installed. The controller 1062 may comprise processing circuitry configured to monitor for the signal from the sensing device 1161 and to take the one or more actions described above in response to receiving the signal. In some embodiments, the controller 1062 may be (or may be part of) the control system 1006 described above in relation to FIG. 8A.

The drape installation indicator 1160 may be any device or object whose presence can be sensed by the sensing device 1161 when the drape installation indicator 1160 is within sufficient proximity. For example, the drape installation indicator 1160 may comprise one or more magnets, and the sensing device 1161 may comprise one or more magnetic field sensors, such as a Hall effect sensor, configured to output a signal when a sensed magnetic field strength exceeds a threshold. As another example, the drape installation indicator 1160 may comprise a radio frequency identification (RFID) tag, and the sensing device 1161 may comprise an RFID reader. As another example, the drape installation indicator 1160 may comprise a visual code, such as a linear bar code or matrix bar code (e.g., QR code), and the sensing device 1161 may comprise a visual reader (e.g., bar code reader).

In some embodiments, the drape installation indicator 1160 also conveys identifying information about the drape assembly 1300 to the controller 1062 via the sensing device 1161, in addition to merely indicating the installed state of the drape assembly 1300 by virtue of its presence. For example, the drape installation indicator 1160 comprising magnets may have identification information of the drape assembly 1300 encoded therein by virtue of the number, location, and/or orientation of magnets included in the drape installation indicator 1160. As another example, the drape installation indicator 1160 comprising the RFID tag may store identification information and transmit the information to the sensing device 1161.

FIG. 15 illustrates an embodiment of a sterile drape assembly 1500. The sterile drape assembly 1500 comprises an ISA 1295. The ISA 1295 is similar to the ISA 195 described above, but instead of being a separate part that is installed over and/or coupled to the sterile drape during installation of the drape, in this embodiment the ISA 1295 is part of the sterile drape 1500. The ISA 1295 comprises intermediate couplers 1296 configured to interface with couplers of the instrument 196 and couplers of the interface 188 of the instrument manipulator mount 187 to transmit drive forces from the manipulator 180 to the instrument 196. In some embodiments, the ISA 1295 is coupled with the sterile drape body 1504 during manufacture. For example, the ISA 1295 may be joined to the sterile drape body 1504 by a heat based joining technique (e.g., welding, heat staking), an adhesive, mechanical fasteners, or any other joining technique. The ISA 1295 may be joined to the drape body 1504 over an opening in the second end 120, similar to the hole 1298 described below. The ISA 1295 is joined to the second end 120 at a location that will correspond to the interface 188 of the instrument manipulator mount 187 when the partially inverted drape assembly 1500 is installed on the manipulator 180. The ISA 1295 is contained within the enclosed interior volume 132 of the drape body 1504 when the drape assembly 1500 is in the initial state (see FIG. 1A) and when the drape assembly is in the partially inverted state (see FIG. 15). When the drape assembly is in the fully inverted state, the ISA 1295 becomes exposed to the environment exterior of the drape assembly 1500, similar to the state illustrated in FIG. 8G.

FIG. 16 illustrates an embodiment of a sterile drape assembly 1600 comprising a hole 1298 in the second end 120 of the drape body 1604. The sterile drape assembly 1600 is configured to have an ISA 195 coupled thereto during installation of the drape assembly 1600, as described above, with the hole 1298 being ultimately covered by the ISA 195, and therefore the hole 1298 (once covered by the ISA 195) does not compromise the sterile barrier established by the sterile drape assembly 1600. The ISA 195 is coupled to the drape body 1604 over the hole 1298, such that the couplers of the ISA 195 are positioned over the hole 1298. This may allow the couplers of the ISA 195, instrument 196, and/or manipulator 198 to engage one another through the hole 1298 without the drape assembly 1600 interfering therewith. This may, in some embodiments, prevent wear and tear on the drape assembly 1600 and/or prevent interference of the drape assembly 1600 with operation of the couplers. In some embodiments, a rim 1299 around the hole 1298 comprises a stiffening member or other engagement feature to facilitate secure coupling between the ISA 195 and the drape body 1604. In some embodiments, the rim 1299 also comprises sealing features, such as a gasket, that facilitates sealing of the drape body 1604 relative to the ISA 195 around the hole 1298 when the ISA 195 is coupled with the drape body 1604. Thus, the ISA 195 and drape assembly 1600, although initially separate pieces, may be coupled together during installation of the ISA 195 or drape assembly 1600 to form an assembly that establishes a continuous sterile barrier over the manipulator 180 notwithstanding the hole 1298. When the ISA 195 is assembled with the drape assembly 1600 in the installed state, the assembly of the drape assembly 1600 with the ISA 195 may be functionally similar to the drape assembly 1500.

FIGS. 17A-17D and 18A-18D illustrate two methods of re-configuring the drape assembly 100 from the initial state (see FIGS. 17A and 18A) to the partially inverted state (see FIGS. 17D and 18D). In a first approach, illustrated in FIGS. 17A-17D, the sterile drape assembly 100 begins in the initial state with the surface 101 facing an exterior environment. The surface 101 will also face the exterior environment when the drape assembly 100 is in the partially inverted state. To transition from the initial state (FIG. 17A) to the partially inverted state (FIG. 17D), the second end 120 of the drape assembly 100 can be pushed toward and into the interior of the drape assembly 100, as indicated by the arrows in FIGS. 17A-17C, such that the portion 108 begins to invert and folds back into the interior of the drape assembly 100, as shown in FIGS. 17B-17D. The second end 120 pushed along until the second end 120 is near the first end 110, as shown in FIG. 17D. In this state, the portion 108 is surrounded by the portion 106, and thus the partially inverted state is obtained.

In a second approach, illustrated in FIGS. 18A-18D, the sterile drape assembly 100 begins in the initial state with the surface 102 facing an exterior environment, instead of the surface 101. To transition from the initial state of the embodiment of FIG. 18A to the partially inverted state of FIG. 18D, which is the same state as the partially inverted states of FIGS. 1B and 2B, in this approach, the first end 110 of the drape body 104 and is folded back over itself, as indicated by the arrows in FIG. 18B. To allow this, the closure mechanism 150 may be in the open state during this process. The first end 110 is pulled along the exterior facing surface 102 of the drape body 104 such that the portion 106 of the drape body 104 ultimately begins to cover and surround the portion 108 of the drape body 104. When the first end 110 has moved past the second end 120 and the portion 106 fully covers the portion 108, then the closure mechanism 150 can be closed and the partially inverted state is obtained, as shown in FIG. 18D. Because the closure mechanism 150 is opened when converting from the initial state to the partially inverted state and then closed in the partially inverted state, in this embodiment the closure mechanism 150 may be a reversible closure mechanism 150.

Herein and in the claims, the partially inverted state may be described in terms of a state in which the portion 106 is folded over and covering the portion 108. This should be understood as referring to a present state of the sterile drape assembly, and not to the manner in which the drape assembly 100 was configured into the partially inverted state. Specifically, the references herein to the portion 106 being “folded over” the portion 108 does not imply that the partially inverted state was achieved by folding the portion 106 over the portion 108. Instead, “folded over” refers to a state or configuration in which the portion 106 covers, surrounds, and overlaps the portion 108 as described above with reference to FIGS. 1B, 2, 17D, and 18D, and this state can be achieved by either folding the portion 106 over the portion 108 or folding the portion 108 into the portion 106—in either case, the end result is a state with the portion 106 folded over the portion 108.

The embodiments described herein may be well suited for use in any of a variety of medical procedures for which sterility of equipment is desired, as described above. Such procedures could be performed, for example, on human patients, animal patients, human cadavers, animal cadavers, and portions or human or animal anatomy. Medical procedures as contemplated herein include any of those described herein and include, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments may also be used for benchtop procedures on non-living material and forms that are not part of a human or animal anatomy. Moreover, some embodiments are also suitable for use in non-medical applications, such as industrial robotic uses, and sensing, inspecting, and/or manipulating non-tissue work pieces. In non-limiting embodiments, the techniques, methods, and devices described herein may be used in, or may be part of, a computer-assisted surgical system employing robotic technology such as the da Vinci® Surgical Systems commercialized by Intuitive Surgical, Inc., of Sunnyvale, California. Those skilled in the art will understand, however, that aspects disclosed herein may be embodied and implemented in various ways and systems, including manually operated instruments and computer-assisted, teleoperated systems, in both medical and non-medical applications. Reference to the daVinci® Surgical Systems are illustrative and not to be considered as limiting the scope of the disclosure herein.

As used herein and in the claims, terms such as computer-assisted manipulator system, teleoperable manipulator system, or manipulator system, should be understood to refer broadly to any system comprising one or more controllable kinematic structures (“manipulators”) comprising one or more links coupled together by one or more joints that can be operated to cause the kinematic structure to move. Such systems may occasionally be referred to in the art and in common usage as robotically assisted systems or robotic systems. The manipulators may have an instrument permanently or removably mounted thereto and may move and operate the instrument. The joints may be driven by drive elements, which may utilize any convenient form of motive power, such as but not limited to electric motors, hydraulic actuators, servomotors, etc. The operation of the manipulator may be controlled by a user (for example through teleoperation), by a computer automatically (so-called autonomous control), or by some combination of these. In examples in which a user controls at least some of the operations of the manipulator, an electronic controller (e.g., a computer) may facilitate or assist in the operation. For example, the electronic controller may “assist” a user-controlled operation by converting control inputs received from the user into electrical signals that actuate drive elements to operate the manipulators, providing feedback to the user, enforcing safety limits, and so on. The term “computer” as used in “computer-assisted manipulator systems” refers broadly to any electronic control device for controlling, or assisting a user in controlling, operations of the manipulator, and is not intended to be limited to things formally defined as or colloquially referred to as “computers.” For example, the electronic control device in a computer-assisted manipulator system could range from a traditional “computer” (e.g., a general-purpose processor plus memory storing instructions for the processor to execute) to a low-level dedicated hardware device (analog or digital) such as a discrete logic circuit or application specific integrated circuit (ASIC), or anything in between. Further, manipulator systems may be implemented in a variety of contexts to perform a variety of procedures, both medical and non-medical. Thus, although some examples described in greater detail herein may be focused on a medical context, the devices and principles described herein are also applicable to other contexts, such as industrial manipulator systems.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes may be made without departing from the spirit and scope of this description and the claims. In some instances, well-known circuits, structures, and techniques have not been shown or described in detail in order not to obscure the embodiments. Like numbers in two or more figures represent the same or similar elements.

Further, the terminology used herein to describe aspects of the invention, such as spatial and relational terms, is chosen to aid the reader in understanding example embodiments of the invention but is not intended to limit the invention. For example, spatially terms—such as “beneath”, “below”, “lower”, “above”, “upper”, “proximal”, “distal”, “up”, “down”, and the like—may be used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein may need to be interpreted differently in that different reference frame. For example, the direction referred to as “up” in relation to one of the figures may correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items may be posed differently.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

As used herein, “proximal” and “distal” are spatial/directional terms that describe locations or directions based on their relationship to the two ends of a kinematic chain. “Proximal” is associated with the end of the kinematic chain that is closer to the base or support of the chain, while “distal” is associated with the opposite end of the kinematic chain, which often comprises an end effector of an instrument. Thus, a “proximal” portion of a component is a portion that is relatively closer to the base of the kinematic chain than other portions of the component. Conversely, a “distal” portion of a component is relatively farther from the base of the kinematic chain than other portions of the component. The terms closer and farther as used above refer to proximity along the kinematic chain, rather than absolute distance. “Proximal” and “distal” directions are directions that point generally from a given location along a kinematic chain towards a more proximal location along the kinematic chain, or from the given location towards a more distal location along the kinematic chain, respectively.

Unless otherwise noted herein or implied by the context, when terms of approximation such as “substantially,” “approximately,” “about,” “around,” “roughly,” and the like, are used in conjunction with a stated numerical value, property, or relationship, such as an end-point of a range or geometric properties/relationships (e.g., parallel, perpendicular, straight, etc.), this should be understood as meaning that mathematical exactitude is not required for the value, property, or relationship, and that instead a range of variation is being referred to that includes but is not strictly limited to the stated value, property, or relationship. In particular, the range of variation around the stated value, property, or relationship includes at least any inconsequential variations from the value, property, or relationship, such as variations that are equivalents to the stated value, property, or relationship. The range of variation around the stated value, property, or relationship also includes at least those variations that are typical in the relevant art for the type of item in question due to manufacturing or other tolerances. Furthermore, the range of variation also includes at least variations that are within +5% of the stated value, property, or relationship. Thus, for example, a line or surface may be considered as being “approximately parallel” to a reference line or surface if any one of the following is true: the smallest angle between the line/surface and the reference is less than or equal to 4.5° (i.e., 5% of) 90°, the angle is less than or equal to manufacturing or other tolerances typical in the art, or the line/surface as constituted is functionally equivalent to the line/surface if it had been perfectly parallel.

As used herein, “transverse” refers to a positional relationship of two items in which one item is oriented crosswise at an angle relative to the other item, such as being substantially or generally perpendicular to the other item. As used herein, “transverse” includes, but does not require, an exactly perpendicular relationship. For example, unless otherwise noted herein or implied by the context, “transverse” may include at least positional relationships in which one item is oriented at an angle between 45° and 135° relative to the other item.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. A sterile drape assembly for draping equipment to create a sterile barrier around the equipment, the sterile drape assembly comprising:
a hollow elongated drape body extending between a first end and a second end, the drape body comprising:
a first drape portion comprising the first end; and
a second drape portion comprising the second end; and
a closure mechanism releasably closing or configured to releasably close the first end of the drape body;
wherein in a partially inverted state of the sterile drape assembly:
the first drape portion is folded back over and covers the second drape portion;
the closure mechanism releasably closes the first end; and
the second drape portion defines a partially enclosed interior volume configured to receive at least a first portion of the equipment.

2. The sterile drape assembly of claim 1,
wherein the drape body has a first surface and a second surface opposite the first surface, and
in the partially inverted state of the sterile drape assembly:
a portion of the first surface that is in the second drape portion faces the partially enclosed interior volume, and
a portion of the first surface that is in the first drape portion faces away from the partially enclosed interior volume.

3. The sterile drape assembly of claim 2,
wherein in the partially inverted state of the sterile drape assembly, a portion of the second surface that is in the first drape portion faces a portion of the second surface that is in the second drape portion.

4. The sterile drape assembly of claim 3,
wherein, in the partially inverted state of the sterile drape assembly:
the second end of the drape body is closed and the closure mechanism is releasably closed such that a fully enclosed internal volume is defined in the drape body, the fully enclosed internal volume being distinct from the partially enclosed internal volume; and
the second surface faces into the fully enclosed internal volume.

5. The sterile drape assembly of claim 4,
wherein in a fully inverted state of the sterile drape assembly:
the closure mechanism is released and the first end of the drape body is open;
the second end of the drape body is closed;
the first and second drape portions of the drape body are positioned sequentially adjacent one another along a length of the drape body and not overlapping; and
the partially enclosed internal volume is extended so as to receive at least the first portion and a second portion of the equipment to create a sterile barrier around the first and second portions of the equipment.

6. The sterile drape assembly of claim 5,
wherein in the fully inverted state of the sterile drape assembly:
the second surface faces outwardly into an exterior environment; and
the first surface faces inwardly into the partially enclosed internal volume.

7. The sterile drape assembly of claim 5,
wherein the equipment comprises a manipulator of a teleoperable medical system, the first portion of the equipment comprises an instrument manipulator mount of the manipulator, and the second portion of the equipment comprises a portion of the manipulator proximal of the instrument manipulator mount.

8. The sterile drape assembly of claim 4,
wherein in a fully inverted state of the sterile drape assembly:
the second surface faces outwardly into an exterior environment; and
the first surface faces inwardly into the partially enclosed internal volume.

9. The sterile drape assembly of claim 2,
wherein in a fully inverted state of the sterile drape assembly:
the second surface faces outwardly into an exterior environment; and
the first surface faces inwardly into the partially enclosed internal volume.

10. The sterile drape assembly of claim 1,
wherein the drape body has a first surface and a second surface opposite the first surface, and
in the partially inverted state of the sterile drape assembly:
the first surface is exposed to an exterior environment; and
the second surface is enclosed within the sterile drape assembly and not exposed to the exterior environment.

11. The sterile drape assembly of claim 10,
wherein in a fully inverted state of the sterile drape assembly:
the second surface faces outwardly into the exterior environment; and
the first surface faces inwardly into the partially enclosed internal volume.

12. The sterile drape assembly of claim 1,
wherein the drape body has a first surface and a second surface opposite the first surface, and
in the partially inverted state of the sterile drape assembly, a portion of the second surface that is in the first drape portion faces a portion of the second surface that is in the second drape portion.

13. The sterile drape assembly of claim 12,
wherein in a fully inverted state of the sterile drape assembly:
the second surface faces outwardly into an exterior environment; and
the first surface faces inwardly into the partially enclosed internal volume.

14. The sterile drape assembly of claim 1,
wherein in a fully inverted state of the sterile drape assembly:
the closure mechanism is released and the first end of the drape body is open;
the second end of the drape body is closed;
the first and second drape portions of the drape body are positioned sequentially adjacent one another along a length of the drape body and not overlapping; and
the partially enclosed internal volume is extended so as to receive at least the first portion and a second portion of the equipment to create a sterile barrier around the first and second portions of the equipment.

15. The sterile drape assembly of claim 1, wherein the closure mechanism comprises a reversible closure mechanism.

16. (canceled)

17. (canceled)

18. The sterile drape assembly of claim 1, wherein the closure mechanism comprises a non-reversible closure mechanism.

19. (canceled)

20. (canceled)

21. The sterile drape assembly of claim 1, further comprising a hold-open device configured to urge the first end of the drape body to remain in an open configuration on condition of the closure mechanism being open.

22. The sterile drape assembly of claim 1, further comprising wiper device configured to, on condition of the closure mechanism being open, hold a rim of an opening in the first end away from the second portion while the first end is moved along the second portion to uncover the second portion.

23. The sterile drape assembly of claim 1, wherein the drape body further comprises a pleated portion at the first end and extending past the closure mechanism, wherein in the partially inverted state the pleated portion is tucked into a fully enclosed interior volume defined by the drape body.

24. (canceled)

25. The sterile drape assembly of claim 1, further comprising a drape installation indicator that is sensible by a sensing device of the equipment, the drape installation indicator being positioned on the drape body so as to be in sensing proximity to the sensing device in a fully installed state of the sterile drape assembly on the equipment.

26. The sterile drape assembly of claim 1, wherein the second end is closed.

27-45. (canceled)
